(19) United States
(12) Patent Application Publication
(10) Pub. No.: US 2016/0251610 A1
(43) Pub. Date: Sep. 1, 2016

(54) MICROBES WITH CONTROLLED ADHESIVE PROPERTIES

(71) Applicants: Michael L. Fisher, Tempe, AZ (US); Roy Curtiss, III, Gainesville, FL (US); Rebecca Custer Allen, Tempe, AZ (US)

(72) Inventors: Michael L. Fisher, Tempe, AZ (US); Roy Curtiss, III, Gainesville, FL (US); Rebecca Custer Allen, Tempe, AZ (US)

(73) Assignee: Arizona Board of Regents on Behalf of Arizona State University, Tempe, AZ (US)

(21) Appl. No.: 15/158,343

(22) Filed: May 18, 2016

Related U.S. Application Data
(63) Continuation of application No. 14/406,461, filed on Dec. 8, 2014, now abandoned.
(60) Provisional application No. 61/657,242, filed on Jun. 8, 2012.
(60) Provisional application No. 61/707,427, filed on Sep. 28, 2012.

(51) Int. Cl. C12N 1/20; C12N 15/74

(52) U.S. Cl. CPC C12N 1/20; C12N 15/74

(57) ABSTRACT

The present invention encompasses a phototrophic microorganism with altered and controlled adhesive properties.

### CROSS REFERENCE TO RELATED APPLICATIONS

This application is a continuation of U.S. application Ser. No. 14/406,461, filed Dec. 8, 2014, which claims the benefit of PCT Application PCT/US2013/044978, filed Jun. 10, 2013, which claims the benefit of U.S. Provisional Application No. 61/707,427, filed Sep. 28, 2012 and U.S. Provisional Application No. 61/657,242, filed Jun. 8, 2012, each of the disclosures of which is hereby incorporated by reference in its entirety.

### GOVERNMENTAL RIGHTS

This invention was made with government support under DE-AR0000011 awarded by the Department of Energy. The government has certain rights in the invention.

### FIELD OF THE INVENTION

The invention encompasses a recombinant microorganism capable of controlled adhesive properties to control biofouling of bioreactors, facilitate generation of efficient biofilm bioreactors, and facilitate harvesting of the microorganism to decrease processing and extraction costs of industrially valuable products produced by the microorganism.

### BACKGROUND OF THE INVENTION

Microorganisms are increasingly being used to manufacture a number of highly valuable products ranging from fuels to pharmaceuticals, including the majority of antibiotics. To manufacture the valuable products using microorganisms, the organisms are grown and harvested, and then the products are extracted from the biomass or the culture medium. While microorganisms are highly efficient at synthesizing the valuable products, costs associated with culture and harvesting of the microorganisms continue to be a barrier to lowering production costs of the valuable products. For instance, in addition to biofouling of bioreactors during culture, biomass harvesting and dewatering remains the most energy intensive step in large-scale biofuel production in photosynthetic microorganisms.

Hence, there is a need in the art for methods of efficiently culturing and harvesting microbes capable of producing valuable products such as biofuels, biofuel precursors, nutritionals or other value added products, while decreasing costs associated with culture, harvesting, and extraction and/or recovery.

### SUMMARY OF THE INVENTION

One aspect of the present invention encompasses a genetically modified phototrophic microorganism capable of controlled adhesion. The microorganism comprises a recombinant nucleic acid construct, wherein the nucleic acid construct comprises an inducible promoter operably-linked to a nucleic acid encoding an adhesion protein. The microorganism is capable of regulated expression of the nucleic acid encoding the adhesion protein. Controlled adhesion of the microorganism may be by expressing the nucleic acid encoding the adhesion protein in the microorganism to enhance the adhesion of the microorganism, by reducing the expression of the nucleic acid encoding the adhesion protein to reduce the adhesion of the microorganism, or by reducing the expression of the nucleic acid encoding the adhesion protein to enhance the adhesion of the microorganism.

Another aspect of the invention encompasses a genetically modified phototrophic microorganism lacking a function encoded by an adhesion protein. The adhesion protein may be selected from the group consisting of the protein encoded by the sll1581 nucleic acid sequence of Synechocystis, the protein encoded by the pilC nucleic acid sequence of Synechocystis, the protein encoded by the sll1951 nucleic acid sequence of Synechocystis, the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

Yet another aspect of the invention comprises a method for controlling adhesion of a phototrophic microorganism. The method comprises introducing into the microorganism a recombinant nucleic acid construct, wherein the nucleic acid construct comprises an inducible promoter operably-linked to a nucleic acid encoding an adhesion protein. The microorganism is capable of regulated expression of the nucleic acid encoding the adhesion protein. Controlled adhesion of the microorganism may be by expressing the nucleic acid encoding the adhesion protein in the microorganism to enhance the adhesion of the microorganism, by reducing the expression of the nucleic acid encoding the adhesion protein to reduce the adhesion of the microorganism, or by reducing the expression of the nucleic acid encoding the adhesion protein to enhance the adhesion of the microorganism.

Still another aspect of the invention comprises a method of reducing the adhesion of a phototrophic microorganism. The method comprises removing from the microorganism an adhesion function of an adhesion protein selected from the group consisting of the protein encoded by the sll1581 nucleic acid sequence of Synechocystis, the protein encoded by the pilC nucleic acid sequence of Synechocystis, and the protein encoded by the sll1951 nucleic acid sequence of Synechocystis, wherein removing the function of the adhesion protein reduces the adhesion of the microorganism.

Another aspect of the invention comprises a method of enhancing the adhesion of a phototrophic microorganism. The method comprises removing from the microorganism an adhesion function of an adhesion protein selected from the group consisting of the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis, wherein removing the function of the adhesion protein enhances the adhesion of the microorganism.

An additional aspect of the invention comprises a method of harvesting a phototrophic microorganism. The method comprises introducing into the microorganism a nucleic acid comprising an inducible promoter operably-linked to a nucleic acid encoding an adhesion protein, and culturing the microorganism. The promoter is then induced to express the nucleic acid encoding the adhesion protein, and the microorganisms in the culture are allowed to autoagglutinate. The autoagglutinated biomass is then collected.

### BRIEF DESCRIPTION OF THE FIGURES

The application file contains at least one photograph executed in color. Copies of this patent application publication with color photographs will be provided by the Office upon request and payment of the necessary fee.

FIG. 1 depicts a schematic diagram of microbial autoaggregation and biomass capture. This method allows for biomass collection in the absence of energy intensive centrifugation. Retention of growth media and water is another feature of sustainability.

FIG. 2A, FIG. 2B and FIG. 2C depict images showing characteristics of autoagglutinating strain SD342 (P_{cmpA}::tibA Kan^{R}). (FIG. 2A) SD342 grown with atmospheric CO₂ to mid-log phase (OD₇₃₀ 0.6). (FIG. 2B) SD342 after 48 hours of CO₂ starvation. Black arrow indicates autoagglutinated biomass. (FIG. 2C) depicts an image showing characteristics of autoagglutinating strain SD1014 (SD1000 ΔnrsAB::P_{nrsB} YadA) grown to stationary phase with increasing amount of inducer (NiCl₂).

FIG. 3A and FIG. 3B depict an image and a graph showing autoagglutination of wild-type or isogenic mutants expressing the: aipA, taaP, tibA, or yadA genes encoding adhesion proteins. (FIG. 3A) Strains were grown in 20 ml of BG-11 at 30° C. with 50 μmol photons/sec/m² to an ˜OD730 of ˜0.6. All cultures were then induced with 6 μM NiCl₂ for 48 hours. Photographs were taken of each strain at the indicated time points. (FIG. 3B) Quantification of autoagglutination and precipitation from the culture medium of wild-type (circles) or isogenic strains expressing aipA (squares), taaP (triangles), tibA (upside-down triangles) or yadA (diamonds). The optical density was measured by removing 1 ml from the top 1 cm of each culture at the time points indicated and measuring the optical density (OD₇₃₀).

FIG. 4A depicts a plot showing biofilm formation (biofouling) by wild-type strains of Synechocystis PCC 6803, and lack of biofilms (biofouling) by mutant strains of Synechocystis PCC 6803. CV=crystal violet, and FIG. 4B depicts a plot showing hyper-biofilm formation for mutant and wild-type strains of Synechocystis PCC 6803 measured by crystal violet absorbance. CV=crystal violet. White bars indicate bacterial cell density as measured by optical density. Black bars indicate the adherence of these strains to culture tubes.

FIG. 5 depicts a plasmid map of pψ508

FIG. 6 depicts a plasmid map of pψ509

FIG. 7 depicts a plasmid map of pψ512

FIG. 8 depicts a plasmid map of pψ513

FIG. 9 depicts a plasmid map of pψ560

FIG. 10 depicts a plasmid map of pψ561

FIG. 11 depicts a plasmid map of pψ588

FIG. 12 depicts a plasmid map of pψ630

FIG. 13 depicts a plasmid map of pψ634

FIG. 14 depicts a plasmid map of pψ635

FIG. 15 depicts a plasmid map of pψ636

FIG. 16 depicts a plasmid map of pψ637

FIG. 17 depicts a plasmid map of pψ540

### DETAILED DESCRIPTION OF THE INVENTION

A phototrophic microorganism with altered adhesive properties has been developed. Using a microorganism of the invention, it is now possible to control biofilm generation and flocculation of microorganisms grown in culture. Advantageously, microorganisms of the invention may be used to increase the efficiency of generating valuable products in microorganisms by facilitating harvesting and preventing biofouling of bioreactors. In addition, such microorganisms may be used to generate highly efficient immobilized cell (thin film) bioreactors. A recombinant microorganism of the invention and methods of using such a recombinant microorganism are described below.

### I. Recombinant Microorganism

One aspect of the present invention provides a recombinant microorganism with altered adhesive properties. Importantly, depending on the desired characteristics of a microorganism of the invention, adhesion may be diminished (reduces biofilm formation, for instance) or enhanced (induces autoagglutination or induces biofilm formation, for instance). According to the invention, the adhesive properties of a microorganism may be altered by A) reducing the expression of a nucleic acid encoding an endogenous adhesion protein, by B) regulating the synthesis of an endogenous adhesion protein, or by C) regulating the expression of a nucleic acid encoding an exogenous adhesion protein. As used herein, “endogenous to a microorganism” refers to a nucleic acid sequence or a protein that is typically present in the wild-type microorganism, while “exogenous to a microorganism” refers to a nucleic acid sequence or protein that is not typically present in the wild-type microorganism.

### (a) Microorganism

A microorganism of the invention may be any phototrophic or photosynthetic microorganism that may be used to produce a valuable product. As used herein, the terms “photosynthetic microorganism” or “phototrophic microorganism” may be used interchangeably, and refer to any microorganism capable of using photons to acquire energy. A phototrophic microorganism may be a bacterium, an alga, or a phytoplankton. In some embodiments, a phototrophic microorganism of the invention is a bacterium. Non-limiting examples of phototrophic bacteria that may be used to produce a valuable product may include species in the genera Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Pleurocapsa, Arthrospira, Borzia, Crinalium, Geitlerinema, Halospirulina, Leptolyngbya, Limnothrix, Lyngbya, Microcoleus, Oscillatoria, Planktothrix, Prochlorothrix, Pseudanabaena, Spirulina, Starria, Symploca, Trichodesmium, Tychonema, Anabaena, Anabaenopsis, Aphanizomenon, Calothrix, Cyanospira, Cylindrospermopsis, Cylindrospermum, Nodularia, Nostoc, Rivularia, Scytonema, Tolypothrix, Chlorogloeopsis, Fischerella, Geitleria, lyengariella, Nostochopsis, and Stigonema of cyanobacteria.

In other embodiments, a phototrophic microorganism of the invention may be a eukaryotic alga. Non-limiting examples of an alga that may be used for the invention may include an alga belonging to the groups archaeplastida such as rhodophyta (Red algae), chlorophyta (Green algae), or glaucophyta; rhizaria or excavata such as chlorarachniophytes and euglenids; heterokonts such as bacillariophyceae (Diatoms), axodine, bolidomonas, eustigmatophyceae, phaeophyceae (Brown algae), chrysophyceae (Golden algae), raphidophyceae, synurophyceae and xanthophyceae (Yellow-green algae); cryptophyta; dinoflagellates; and haptophyta. In preferred embodiments, a microorganism is a phytoplankton, which includes diatoms, dinoflagellates and coccolithophores, in addition to cyanobacteria and algae.

In preferred embodiments, a microorganism of the invention may be a cyanobacterium. Non-limiting examples of cyanobacteria that may be used in the invention may include cyanobacteria belonging to the order Chroococcales, cyanobacteria belonging to the order Nostocales, and cyanobacteria belonging to the order Stigonematales. In some embodiments, a cyanobacterium belongs to the order Nostocales. In other embodiments, a cyanobacterium belongs to the order Stigonematales. In yet other embodiments, a cyanobacterium belongs to the order Chroococcales. In a preferred embodiment, a bacterium is derived from the genus Synechocystis. For instance, a bacterium of the invention may be derived from Synechocystis PCC sp. 6803.

### (b) Adhesion Protein

The term “adhesion protein” as used herein, refers to any protein that may alter the adhesive properties of a microorganism when the synthesis of the adhesion protein in the microorganism is altered. As used herein, “alter the adhesive properties of a microorganism” or “alter adhesion of a microorganism” are used interchangeably and refer to altering autoagglutination (adhesion between two or more microorganisms) or adhesion of a microorganism to a solid surface (adhesion between a microorganism and a solid surface). To determine if a particular protein is an adhesion protein, one of skill in the art may alter the synthesis of the protein and use assays commonly known in the art to determine if there is a change in the adhesive properties of the microorganism. As used herein, “adhesion function” refers to the function of an adhesion protein. Such function may include up or down regulation of nucleic acid expression that impacts adhesion, up or down regulation of protein synthesis that impacts adhesion, or up or down regulation of nucleic acid or protein half-life that impacts adhesion.

Generally speaking, adhesion proteins fall into three categories: A) any protein that, when expression of a nucleic acid encoding the adhesion protein is reduced in a microorganism, may lead to defective adhesion of the microorganism; B) any protein that, when expression of a nucleic acid encoding the adhesion protein is reduced in a microorganism, may lead to enhanced adhesion of the microorganism; and C) any protein that, when synthesized by a microorganism, may increase adhesion of the microorganism.

The terms “defective adhesion”, “reduced adhesion”, and “decreased adhesion” may be used interchangeably and refer to lower levels of adhesion of a microorganism comprising altered synthesis of adhesion protein, when compared to the levels of adhesion of the microorganism when the synthesis of the adhesion protein is not altered. In general, when adhesion is reduced in a microorganism, the microorganism may not be capable of autoagglutination or adhesion to a solid surface. The terms “enhanced adhesion”, “increased adhesion”, and “improved adhesion” may be used interchangeably and refer to higher levels of adhesion of the microorganism comprising the altered synthesis of an adhesion protein, when compared to the levels of adhesion of the microorganism when the synthesis of the adhesion protein is not altered. In general, when adhesion is enhanced in a microorganism, the microorganism may be capable of autoagglutination or adhesion to a solid surface.

An adhesion protein may be an adhesion protein present on the cell surface of the microorganism, directly providing the adhesive property to the microorganism. Alternatively, an adhesion protein may affect the expression of cell surface molecules that provide the adhesive properties to the microorganism. For instance, an adhesion protein may be a cytoplasmic protein capable of regulating expression of a cell surface adhesive molecule. Non-limiting examples of adhesive molecules may include exopolysaccharides, lipoproteins, glycoproteins, and cell surface adhesive proteins as described above.

An adhesion protein may be an endogenous protein of a microorganism of the invention, or an exogenous protein introduced into a microorganism by the introduction into the microorganism of a nucleic acid encoding that adhesion protein. An adhesion protein of the invention may be a naturally occurring adhesion protein from a microorganism naturally capable of adhesion. Alternatively, an adhesion protein of the invention may be a protein that was genetically engineered to provide an adhesion property to a microorganism.

An adhesion protein may be a single protein capable of altering adhesion of a microorganism, or may comprise two or more proteins that, when synthesized in a microorganism, may induce adhesion of the microorganism.

In some embodiments, an adhesion protein comprises two or more individual proteins that, when synthesized in a microorganism, may induce adhesion of the microorganism. In one embodiment, an adhesion protein comprises two, three, four, five or more proteins that, when synthesized in a microorganism, may induce adhesion of the microorganism. In a preferred embodiment, an adhesion protein comprises two proteins that, when synthesized in a microorganism, may induce adhesion of the microorganism. Non-limiting examples of two proteins that, when synthesized in a microorganism may induce adhesion of the microorganism may include invasin and α₁β₁ integrin, an antibody or an antibody fragment and its corresponding antigen, a phage tail fiber and its corresponding receptor such as the lambda phage tail fiber and its receptor encoded by the lamB nucleic acid sequence of E. coli, T4 tail fiber and its receptor encoded by the fadL nucleic acid sequence of E. coli. In an exemplary embodiment, adhesion proteins are the lambda phage tail fiber and its receptor encoded by the lamB nucleic acid sequence of E. coli, wherein the lambda phage tail fiber is operably linked to the protein encoded by the slr1951 nucleic acid sequence for localization on the S layer.

In one embodiment, two adhesion proteins may be synthesized in the same microorganism. In an alternative embodiment, each of two adhesion proteins may be synthesized in distinct populations of a microorganism and, when the distinct populations of the microorganism are in the same culture, may induce autoagglutination. In preferred embodiments, two adhesion proteins may be synthesized in the same microorganism.

In some embodiments, an adhesion protein is a single protein capable of altering adhesion of the microorganism. Non limiting examples of a protein capable of altering adhesion of the microorganism include toxin-coregulated pilus (TCP) of Vibrio cholera, PapG of E. coli, SfaS of E. coli, FimH of E. coli, HifE of Haemophilus influenzae, PrsG of E. coli, MrkD of Klebsiella pneumonia, FHA of Bordetella pertussis, Pertactin of Bordetella pertussis, HMW1/HMW2 of Haemophilus influenzae, Hia of Haemophilus influenzae, Le^{b}-binding adhesin of Bordetella pertussis, Ag I/II of Streptococcus mutans, SpaP of Streptococcus mutans, P1 of Streptococcus mutans, PAc of Streptococcus mutans, SspA of Streptococcus mutans, SspB of Streptococcus mutans, SpaA of Streptococcus sobrinus, PAg of Streptococcus sobrinus, CshA of Streptococcus gordonii, FnbA of Staphylococcus aureus, FnbB of Staphylococcus aureus, Sfbl of Streptococcus pyogenes, protein F of Streptococcus pyogenes, FimA of Streptococcus parasanguis, PsaA of Streptococcus pneumonia, ScaA of Streptococcus gordonii, SsaB of Streptococcus sanguis, EfaA of Enterococcus faecalis, CbpA of Streptococcus pneumonia, SpsA of Streptococcus pneumonia, PbcA of Streptococcus pneumonia, PspC of Streptococcus pneumonia, the protein encoded by the tibA nucleic acid sequence of E. coli, the protein encoded by the yadA nucleic acid sequence of Yersinia species, the protein encoded by the aipA nucleic acid sequence of Proteus mirabilis, the protein encoded by the taaP nucleic acid sequence of Proteus mirabilis, the protein Ag43a encoded by the fluA nucleic acid sequence of E. coli, the protein encoded by the sll1581 nucleic acid sequence of Synechocystis, the protein encoded by the pilC nucleic acid sequence of Synechocystis, the protein encoded by the sll1951 nucleic acid sequence of Synechocystis, the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

In some embodiments, an adhesion protein is a single protein that, when the expression of a nucleic acid sequence encoding the adhesion protein is inactivated (e.g., reduced) in a microorganism, leads to reduced adhesion of the microorganism. Non limiting examples of adhesion proteins that, when inactivated in a microorganism, lead to reduced adhesion of the microorganism, include the protein encoded by the sll1581 nucleic acid sequence of Synechocystis, the protein encoded by the pilC nucleic acid sequence of Synechocystis, and the protein encoded by the sll1951 nucleic acid sequence of Synechocystis. In one embodiment, an adhesion protein that, when inactivated in a microorganism, leads to defective adhesion of the microorganism, is encoded by the sll1581 nucleic acid sequence of Synechocystis. In another embodiment, an adhesion protein that, when inactivated in a microorganism, leads to defective adhesion of the microorganism, is encoded by the pilC nucleic acid sequence of Synechocystis. In an additional embodiment, an adhesion protein that, when inactivated in a microorganism, leads to defective adhesion of the microorganism, is encoded by the sll1951 nucleic acid sequence of Synechocystis.

In other embodiments, an adhesion protein is a single protein that, when the expression of a nucleic acid sequence encoding the adhesion protein is inactivated (e.g., reduced) in a microorganism, leads to enhanced adhesion of the microorganism. Non limiting examples of adhesion proteins that, when inactivated in a microorganism, lead to enhanced adhesion of the microorganism include the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis. slr0977 encodes a membrane permease through which EPS is delivered to the cell membrane. slr0982 encodes an ABC protein, which is believed to provide the energy necessary for EPS transport through the permease encoded by slr0977. slr1610 is a putative methyltransferase that putatively functions in chain termination of carbohydrate elongation. The absence of one or more of these nucleic acid sequences encoding an adhesion protein leads to an enhanced aggregation phenotype in Synechocystis. In one embodiment, an adhesion protein that, when inactivated in a microorganism, leads to enhanced adhesion of the microorganism, is encoded by the slr0977 nucleic acid sequence of Synechocystis. In another embodiment, an adhesion protein that, when inactivated in a microorganism, leads to enhanced adhesion of the microorganism, is encoded by the slr0982 nucleic acid sequence of Synechocystis. In yet another embodiment, an adhesion protein that, when inactivated in a microorganism, leads to enhanced adhesion of the microorganism, is encoded by the slr1610 nucleic acid sequence of Synechocystis.

In certain embodiments, an adhesion protein is a single protein that, when the expression of a nucleic acid sequence encoding the adhesion protein is induced (e.g., synthesized in a microorganism) in a microorganism, may enhance adhesion of the microorganism. Such proteins may include type V adhesins, autotransporter adhesins, adhesins of gram-negative bacteria, and adhesins of gram-positive bacteria. Non-limiting examples of adhesion proteins that, when the expression of a nucleic acid sequence encoding the adhesion protein is induced in a microorganism, may enhance adhesion of the microorganism, include toxin-coregulated pilus (TCP) of Vibrio cholera, PapG of E. coli, SfaS of E. coli, FimH of E. coli, HifE of Haemophilus influenzae, PrsG of E. coli, MrkD of Klebsiella pneumonia, FHA of Bordetella pertussis, Pertactin of Bordetella pertussis, HMW1/HMW2 of Haemophilus influenzae, Hia of Haemophilus influenzae, Le^{b}-binding adhesin of Bordetella pertussis, Ag I/II of Streptococcus mutans, SpaP of Streptococcus mutans, P1 of Streptococcus mutans, PAc of Streptococcus mutans, SspA of Streptococcus mutans, SspB of Streptococcus mutans, SpaA of Streptococcus sobrinus, PAg of Streptococcus sobrinus, CshA of Streptococcus gordonii, FnbA of Staphylococcus aureus, FnbB of Staphylococcus aureus, Sfbl of Streptococcus pyogenes, protein F of Streptococcus pyogenes, FimA of Streptococcus parasanguis, PsaA of Streptococcus pneumonia, ScaA of Streptococcus gordonii, SsaB of Streptococcus sanguis, EfaA of Enterococcus faecalis, CbpA of Streptococcus pneumonia, SpsA of Streptococcus pneumonia, PbcA of Streptococcus pneumonia, PspC of Streptococcus pneumonia, the protein encoded by the tibA nucleic acid sequence of E. coli, the protein encoded by the yadA nucleic acid sequence of Yersinia species, the protein encoded by the aipA nucleic acid sequence of Proteus mirabilis, the protein encoded by the taaP nucleic acid sequence of Proteus mirabilis, and the protein Ag43a encoded by the fluA nucleic acid sequence of E. coli.

In one embodiment, an adhesion protein that, when the expression of a nucleic acid sequence encoding the adhesion protein is induced in a microorganism, leads to enhanced adhesion of the microorganism, is encoded by the yadA nucleic acid sequence of Yersinia species. In another embodiment, an adhesion protein that, when the expression of a nucleic acid sequence encoding the adhesion protein is induced in a microorganism, leads to enhanced adhesion of the microorganism, is encoded by the tibA nucleic acid sequence of E. coli. In yet another embodiment, an adhesion protein that, when the expression of a nucleic acid sequence encoding the adhesion protein is induced in a microorganism, leads to enhanced adhesion of the microorganism, is encoded by the aipA nucleic acid sequence of Proteus mirabilis. In one embodiment, an adhesion protein that, when the expression of a nucleic acid sequence encoding the adhesion protein is induced in a microorganism, leads to enhanced adhesion of the microorganism, is encoded by the taaP nucleic acid sequence of Proteus mirabilis. In one embodiment, an adhesion protein that, when the expression of a nucleic acid sequence encoding the adhesion protein is induced in a microorganism, leads to enhanced adhesion of the microorganism, is the protein Ag43a encoded by the fluA nucleic acid sequence of E. coli. In one embodiment, an adhesion protein that, when the expression of a nucleic acid sequence encoding the adhesion protein is induced in a microorganism, leads to enhanced adhesion of the microorganism, is encoded by the sll1581 nucleic acid sequence of Synechocystis. In one embodiment, an adhesion protein that, when the expression of a nucleic acid sequence encoding the adhesion protein is induced in a microorganism, leads to enhanced adhesion of the microorganism, is encoded by the pilC nucleic acid sequence of Synechocystis. In one embodiment, an adhesion protein that, when the expression of a nucleic acid sequence encoding the adhesion protein is induced in a microorganism, leads to enhanced adhesion of the microorganism, is encoded by the sll1951 nucleic acid sequence of Synechocystis. In a particularly exemplary embodiment, an adhesion protein is encoded by the tibA nucleic acid sequence of E. coli described in Table 3. In another particularly exemplary embodiment, an adhesion protein is encoded by the yadA nucleic acid sequence of E. coli described in Table 3. In yet another particularly exemplary embodiment, an adhesion protein is encoded by the aipA nucleic acid sequence of P. mirabilis described in Table 3. In another particularly exemplary embodiment, the protein is encoded by the taaP nucleic acid sequence P. mirabilis described in Table 3.

An adhesion protein of the invention may be a naturally occurring adhesion protein or an engineered or altered adhesion protein, or a homolog, ortholog, mimic or degenerative variant of an adhesion protein, or it may be an artificial or man-made polypeptide. In some embodiments, the adhesion protein of the invention is a naturally occurring adhesion protein. In exemplary embodiments, an adhesion protein is a naturally occurring adhesion protein encoded by a nucleic acid sequence as in Table 3.

In other embodiments, an adhesion protein of the invention is an artificial adhesion protein. In other embodiments, an adhesion protein of the invention is a naturally occurring adhesion protein with at least one sequence alteration. Such sequence alterations in adhesion proteins are known in the art to greatly alter the adhesive properties of the gene product. An adhesion protein of the invention may comprise 1, 2, 3, 4, 5, 6, 7, 8, 9, 10 or more, or a combination of one or more sequence alterations. In one embodiment, an adhesion protein of the invention is a naturally occurring adhesion protein comprising 1, 2, 3, 4, or 5 sequence alterations. In another embodiment, an adhesion protein of the invention is a naturally occurring adhesion protein comprising 5, 6, 7, 8, 9, 10 or more sequence alterations.

Methods of producing adhesion proteins with sequence alterations are known in the art and may include introducing a deletion, substitution, addition, or insertion into a nucleic acid sequence encoding a protein. Such an alteration may be generated using recombinant techniques to introduce deletions, insertions and point mutations. Non limiting examples of recombinant techniques that may be used to generate altered adhesion proteins may include site-directed mutagenesis, e.g., using nucleic acid amplification techniques such as PCR, the use of either BAL 31 nuclease or exonuclease III for deletion mutagenesis, treatment with mutagens, such as sodium bisulfite, enzymatic incorporation of nucleotide analogs, or misincorporation of normal nucleotides or alpha-thionucleotide by DNA polymerases. Additional information may be found in Sambrook et al., Molecular Cloning: A Laboratory Manual (New York: Cold Spring Harbor Laboratory Press, 1989).

Adhesion proteins may be localized on the surface of the microorganism for adhesion. For instance, adhesion proteins may be localized on pili of a microorganism, or on the S layer of a microorganism. In some embodiments, adhesion proteins are localized on pili of a microorganism. In other embodiments, adhesion proteins are localized on the S layer of a microorganism. For instance, an adhesion protein may be localized on the S layer of a microorganism by operably linking the adhesion protein to a surface protein, protein fragment or peptide localized on the S layer of the microorganism. In an exemplary embodiment, an adhesion protein is encoded by the tibA nucleic acid sequence of E. coli and is localized on the pili of a microorganism.

In some embodiments, a recombinant microorganism of the invention may comprise one, two, three, four, five, six, seven, or eight adhesion proteins. To ensure that the synthesis of an adhesion protein does not result in the premature adhesion of a phototrophic microorganism, a nucleic acid sequence encoding an adhesion protein may be placed under the control of an inducible promoter as described in Section I(c) below.

### (c) Altered Synthesis of Adhesion Protein

According to the invention, adhesive properties of a microorganism may be altered by altering the expression of one or more nucleic acid sequences encoding an adhesion protein, leading to altered synthesis of the adhesion protein. For instance, the adhesive properties of a microorganism may be altered by altering the expression of 1, 2, 3, 4, 5, 6, 7, 8, 9, 10 or more nucleic acid sequences, each encoding an adhesion protein. In some embodiments, the expression of 1, 2, 3, 4 or 5 nucleic acid sequences, each encoding an adhesion protein, is altered. In other embodiments, the expression of 5, 6, 7, 8, 9, or 10 nucleic acid sequences, each encoding an adhesion protein, is altered. In a preferred embodiment, the expression of one nucleic acid sequence encoding an adhesion protein is altered. Altering the expression of a nucleic acid typically comprises reducing the expression of an endogenous nucleic acid encoding an adhesion protein, or regulating the expression of an endogenous or exogenous nucleic acid, as described below.

In some embodiments, adhesive properties of a microorganism may be altered by reducing the expression of an endogenous nucleic acid sequence encoding an adhesion protein (thereby altering the synthesis of the adhesion protein). In one embodiment, expression of an endogenous nucleic acid sequence encoding an adhesion protein is substantially reduced. In a preferred embodiment, expression of an endogenous nucleic acid sequence encoding an adhesion protein is eliminated. Such a reduction in expression may either decrease adhesion (e.g., to reduce biofilm formation in a bioreactor) or increase adhesion (form a biofilm in a biofilm bioreactor).

In one embodiment, expression of an endogenous nucleic acid sequence encoding an adhesion protein is reduced in a microorganism, wherein reducing synthesis of the adhesion protein reduces adhesion of the microorganism. This may lead to reduced biofilm formation, and consequently, reducing biofouling. In a preferred embodiment, expression of an endogenous nucleic acid sequence encoding an adhesion protein is reduced in a Synechocystis microorganism, resulting in defective adhesion of the microorganism. Non limiting examples of adhesion proteins endogenous to a Synechocystis microorganism that, when the synthesis of the adhesion protein is reduced in the microorganism, may produce defective adhesion of the microorganism, include the protein encoded by the sll1581 nucleic acid sequence of Synechocystis, the protein encoded by the pilC nucleic acid sequence of Synechocystis, and the protein encoded by the sll1951 nucleic acid sequence of Synechocystis. In an exemplary embodiment, expression of the protein encoded by the sll1581 nucleic acid sequence is reduced in a Synechocystis microorganism, resulting in defective adhesion of the microorganism. In another exemplary embodiment, expression of the protein encoded by the pilC nucleic acid sequence is reduced in a Synechocystis microorganism, resulting in defective adhesion of the microorganism. In yet another exemplary embodiment, expression of the protein encoded by the sll1951 nucleic acid sequence is reduced in a Synechocystis microorganism, resulting in defective adhesion of the microorganism.

In another embodiment, expression of an endogenous nucleic acid sequence encoding an adhesion protein is reduced in a microorganism, wherein reducing synthesis of the adhesion protein enhances adhesion of the microorganism. This may lead to enhanced biofilm formation, and consequently, facilitate production of thin film bioreactors, or facilitate autoagglutination and harvesting of microorganisms. In a preferred embodiment, expression of an endogenous nucleic acid sequence encoding an adhesion protein is reduced in a Synechocystis microorganism, resulting in enhanced adhesion of the microorganism. Non limiting examples of adhesion proteins endogenous to a Synechocystis microorganism that, when synthesis of the adhesion protein is reduced, may produce enhanced adhesion of the microorganism, include the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis. In an exemplary embodiment, expression of the protein encoded by the slr0977 nucleic acid sequence is reduced in a Synechocystis microorganism, resulting in enhanced adhesion of the microorganism. In another exemplary embodiment, expression of the protein encoded by the slr0982 nucleic acid sequence is reduced in a Synechocystis microorganism, resulting in enhanced adhesion of the microorganism. In yet another exemplary embodiment, expression of the protein encoded by the slr1610 nucleic acid sequence is reduced in a Synechocystis microorganism, resulting in enhanced adhesion of the microorganism.

Methods of reducing the expression of a nucleic acid sequence encoding a protein are known in the art and may include introducing an inactivating mutation in the nucleic acid sequence encoding the protein or in the nucleic acid sequences controlling the synthesis of the protein. Additional information may be found in Sambrook et al., Molecular Cloning: A Laboratory Manual (New York: Cold Spring Harbor Laboratory Press, 1989).

In other embodiments, adhesive properties of a microorganism of the invention may be altered by regulating the expression of an endogenous or exogenous nucleic acid sequence encoding an adhesion protein in the microorganism. The phrase “regulating the expression of a nucleic acid sequence encoding an adhesion protein” refers to expression in a microorganism of a nucleic acid encoding an adhesion protein such that the adhesive properties of the microorganism are altered. In one embodiment, expression of a nucleic acid sequence encoding an adhesion protein is increased in a microorganism. In another embodiment, expression is substantially reduced. In yet another embodiment, expression of a nucleic acid sequence encoding an adhesion protein is modulated in a temporally controlled manner. For instance, expression of a nucleic acid sequence encoding an adhesion protein may be reduced during culture of the microorganism in a bioreactor to prevent biofilm formation that may lead to biofouling of the bioreactor, and subsequently, expression may be increased to induce autoagglutination and facilitate biomass recovery. Alternatively, expression of a nucleic acid sequence encoding an adhesion protein may be increased in a microorganism to generate a thin film bioreactor, and subsequently, expression may be reduced during culture of the microorganism in a bioreactor to prevent biofilm formation that may lead to biofouling of the bioreactor.

In some embodiments, adhesive properties of a microorganism of the invention may be altered by regulating the expression of an endogenous nucleic acid sequence encoding an adhesion protein. Non limiting examples of methods of regulating the expression of an endogenous nucleic acid sequence in a microorganism are known in the art, and may include introducing into the microorganism a nucleic acid construct comprising a nucleic acid sequence encoding the endogenous adhesion protein (e.g., a plasmid), replacing the native promoter of an endogenous nucleic acid encoding an adhesion protein, modifying the native promoter of an endogenous nucleic acid encoding an adhesion protein, or combinations thereof. In one embodiment, adhesive properties of a microorganism may be altered by introducing into the microorganism a nucleic acid construct capable of expressing an endogenous nucleic acid sequence encoding an adhesion protein. Such a nucleic acid construct may be chromosomally integrated, or may be expressed on an extrachromosomal vector. In another embodiment, adhesive properties of a microorganism may be altered by replacing the native promoter of a nucleic acid encoding an adhesion protein with a promoter capable of regulating the expression of the nucleic acid encoding the adhesion protein. As such, adhesive properties of a microorganism may be altered by inducing or repressing the expression from the promoter capable of regulating the expression of the nucleic acid encoding the adhesion protein.

In other embodiments, adhesive properties of a microorganism of the invention may be altered by introducing into the microorganism a nucleic acid construct capable of expressing an exogenous nucleic acid sequence encoding an adhesion protein. Such a nucleic acid construct may be chromosomally integrated, or may be expressed on an extrachromosomal vector.

Methods of making a microorganism of the invention are known in the art. Generally speaking, a microorganism is transformed with a nucleic acid construct of the invention. Methods of transformation are well known in the art, and may include electroporation, natural transformation, and calcium chloride mediated transformation. Methods of screening for and verifying chromosomal integration are also known in the art.

In a preferred embodiment, a microorganism is a photosynthetic cyanobacterium, and a method of making a cyanobacterium of the invention may comprise first transforming the cyanobacterium with a vector comprising, in part, an antibiotic-resistance marker and a negative selection marker. Chromosomal integration may be selected for by selecting for antibiotic resistance. Next, the antibiotic-resistant strain is transformed with a similar vector comprising the target nucleic acids of interest. Chromosomal integration of the target nucleic acids may be selected for by selecting for the absence of the negative marker. For instance, if the negative marker is sacB, then one would select for sucrose resistance. For more details, see Kang et al., J. Bacteriol. (2002) 184(1):307-12, Sun et al., Appl. Environ. Microbiol. (2008) 74:4241-45, and Liu et al., PNAS (2011) 108:6899-6904, hereby incorporated by reference in their entirety.

In other embodiments, the microorganism is a eukaryotic alga. Nucleic acid sequences may be expressed in the nucleus or the plastid of eukaryotic algal cells. As is generally recognized in the art, chloroplasts use bacterial means for expression of nucleic acid sequences and for protein synthesis. As such, methods for regulated or constitutive expression of nucleic acid sequences in algal chloroplasts are as described for expression of nucleic acid sequences in bacteria. Methods of transforming an alga and to express nucleic acid sequences from the nucleus or the plastid of the algal cell are known in the art. For more details, see Wang et al., J. Genet. Genomics (2009) 36:387-398, Radakovits et al., Eukaryotic Cell (2010) 9(4):486-501, Newell et al. (2003) 12:631-634, hereby incorporated by reference in their entirety.

i. Nucleic Acid Constructs

When a nucleic acid construct capable of expressing an endogenous or exogenous nucleic acid sequence encoding an adhesion protein is introduced into a microorganism of the invention, the microorganism may be capable of regulated expression of the nucleic acid encoding an adhesion protein. As such, a microorganism of the invention may comprise a recombinant nucleic acid construct, wherein the nucleic acid construct comprises an inducible promoter operably-linked to a nucleic acid encoding an adhesion protein, wherein the microorganism is capable of regulated synthesis of the adhesion protein. Such a nucleic acid construct may be chromosomally integrated, or may be expressed on an extrachromosomal vector. Each component of the above nucleic acid construct is discussed in more detail below.

Methods of making a nucleic acid construct of the invention are known in the art. Additional information may be found in Sambrook et al., Molecular Cloning: A Laboratory Manual (New York: Cold Spring Harbor Laboratory Press, 1989).

ii. Promoters

A nucleic acid construct of the present invention that expresses a nucleic acid encoding a protein capable of inducing adhesion comprises a promoter. In some embodiments, a protein is synthesized using an inducible promoter operably-linked to a nucleic acid encoding the protein. In other embodiments, a protein is synthesized using a first inducible promoter and a second constitutive promoter operably-linked to a nucleic acid encoding the protein capable of inducing adhesion. When a nucleic acid comprises a first and a second promoter, the promoters may read in opposite directions, or may read in the same direction.

A. First Inducible Promoter

In certain embodiments, a nucleic acid of the invention encompasses an inducible promoter. Non-limiting examples of inducible promoters may include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by absence of small molecules (e.g., CO₂, iron, nitrogen), by metals or metal ions (e.g., copper, zinc, cadmium, nickel), by environmental factors (e.g., heat, cold, stress, light, darkness), and by growth phase. In each of the above embodiments, the inducible promoter is preferably tightly regulated such that in the absence of induction, substantially no transcription is initiated through the promoter. Additionally, induction of a promoter of interest should not typically alter transcription through other promoters. Also, generally speaking, a compound or condition that induces an inducible promoter should not be naturally present in the organism or environment where expression is sought.

In some embodiments, an inducible promoter is induced by limitation of CO₂ supply to a microbacterial culture comprising a microorganism of the invention. By way of non-limiting example, an inducible promoter may be a variant sequence of a Synechocystis PCC 6803 promoter that is up-regulated under conditions of CO₂-limitation. Such a promoter may include the promoter of the cmp nucleic acid sequence, the promoter of the ntp nucleic acid sequence, the promoter of the ndh nucleic acid sequence, the promoter of the sbt nucleic acid sequence, the promoter of the chp nucleic acid sequence, the promoter of the pilA nucleic acid sequence, the promoter of the rbc nucleic acid sequence, the promoter of the gifA nucleic acid sequence, the promoter of the cupA nucleic acid sequence, the promoter of the sll0219 nucleic acid sequence, and the promoter of the sll0217 nucleic acid sequence. In exemplary embodiments, an inducible promoter is the P_{cmp} promoter of the cmp nucleic acid sequence of Synechocystis PCC 6803, wherein the promoter is induced by the lack of CO₂.

In other embodiments, an inducible promoter is induced by iron starvation. By way of non-limiting example, an inducible promoter may be a variant sequence of the promoter of the Synechocystis PCC 6803 isiA gene.

In yet other embodiments, an inducible promoter is induced at the stationary growth phase of a microorganism. By way of non-limiting example, an inducible promoter may be a variant sequence of a Synechocystis PCC 6803 promoter that is up-regulated when the culture enters stationary growth phase. Such a promoter may include the promoter of the isiA nucleic acid sequence, the promoter of the phrA nucleic acid sequence, the promoter of the sigC nucleic acid sequence, the promoter of the sigB nucleic acid sequence, and the promoter of the sigH nucleic acid sequence.

In other embodiments, an inducible promoter is induced by a metal or metal ion. By way of non-limiting example, an inducible promoter may be induced by copper, zinc, cadmium, mercury, nickel, gold, silver, cobalt, and bismuth or ions thereof. In one embodiment, an inducible promoter may be induced by copper or a copper ion. In another embodiment, an inducible promoter may be induced by zinc or a zinc ion. In yet another embodiment, an inducible promoter may be induced by cadmium or a cadmium ion. In still another embodiment, an inducible promoter may be induced by mercury or a mercury ion. In an alternative embodiment, an inducible promoter may be induced by gold or a gold ion. In another alternative embodiment, an inducible promoter may be induced by silver or a silver ion. In yet another alternative embodiment, an inducible promoter may be induced by cobalt or a cobalt ion. In still another alternative embodiment, an inducible promoter may be induced by bismuth or a bismuth ion. In a preferred embodiment, an inducible promoter is induced by nickel or a nickel ion. In an exemplary embodiment, an inducible promoter is induced by a nickel ion, such as Ni²⁺. In another exemplary embodiment, an inducible promoter is the P_{nrsB} nickel inducible promoter from Synechocystis PCC 6803.

When an inducible promoter is induced by a metal or metal ion, expression of a nucleic acid encoding an adhesion protein may be induced by exposing a microbial cell comprising the inducible promoter to a metal or metal ion. A cell may be exposed to a metal or metal ion by adding the metal to the microbial growth media. In certain embodiments, a metal or metal ion added to a bacterial growth media may be efficiently recovered from the media. In other embodiments, a metal or metal ion remaining in a media after recovery does not substantially impede downstream processing of the media or of bacterial gene products.

B. Second Promoter

Certain nucleic acid constructs of the invention may comprise a second promoter. A second promoter may be an inducible promoter, or may be a constitutive promoter. If a second promoter is an inducible promoter, it may or may not be induced by the same compound or condition that induces the first inducible promoter. In one embodiment, the same compound or condition induces both a first and a second inducible promoter. In another embodiment, a first inducible promoter is induced by a different compound or condition than a second inducible promoter. Non-limiting examples of inducible promoters that may be used are detailed in Section I(c)(ii)(A) above.

Constitutive promoters are known in the art. Non-limiting examples of constitutive promoters may include constitutive promoters from Gram-negative bacteria or a bacteriophage propagating in a Gram-negative bacterium. For instance, promoters for genes encoding highly expressed Gram-negative gene products may be used, such as the promoter for Lpp, OmpA, rRNA, and ribosomal proteins. Alternatively, regulatable promoters may be used in a strain that lacks the regulatory protein for that promoter. For instance P_{lac}, P_{tac}, and P_{trc} may be used as constitutive promoters in strains that lack Lacl. Similarly, P22 P_{R} and P_{L} may be used in strains that lack the P22 C2 repressor protein, and λ P_{R} and P_{L} may be used in strains that lack the λ C1 repressor protein. In one embodiment, a constitutive promoter is from a bacteriophage. In another embodiment, a constitutive promoter is from a Salmonella bacteriophage. In yet another embodiment, a constitutive promoter is from a cyanophage. In some embodiments, a constitutive promoter is a Synechocystis promoter. For instance, a constitutive promoter may be the P_{psbAll} promoter or its variant sequences, the P_{rbc} promoter or its variant sequences, the P_{cpc} promoter or its variant sequences, or the P_{mpB} promoter or its variant sequences. In preferred embodiments, a second promoter is the P_{trc} promoter.

In some embodiments, a protein of the invention is synthesized using a first inducible promoter and a second constitutive promoter operably-linked to a nucleic acid encoding an adhesion protein. In a preferred embodiment, a protein of the invention is synthesized using a first inducible promoter induced by the lack of CO₂ and a second constitutive promoter operably-linked to a nucleic acid encoding an adhesion protein. In an exemplary embodiment, a protein of the invention is synthesized using a first P_{cmp} inducible promoter induced by the lack of CO₂ and a second P_{trc} constitutive promoter operably-linked to a nucleic acid encoding an adhesion protein.

In another preferred embodiment, a protein of the invention is synthesized using a first inducible promoter induced by nickel and a second constitutive promoter operably-linked to a nucleic acid encoding an adhesion protein. In an exemplary embodiment, a protein of the invention is synthesized using a first P_{nrsB} inducible promoter induced by nickel and a second P_{trc} constitutive promoter operably-linked to a nucleic acid encoding an adhesion protein.

iii. Additional Components

In certain embodiments, nucleic acids of the invention may further comprise additional components, such as a marker, a spacer domain, and a flanking sequence.

A. markers

In one embodiment, a nucleic acid of the invention comprises at least one marker. Generally speaking, a marker encodes a product that the host cell cannot make, such that the cell acquires resistance to a specific compound, is able to survive under specific conditions, or is otherwise differentiable from cells that do not carry the marker. Markers may be positive or negative markers. In some embodiments, a nucleic acid of the invention may comprise both a positive marker and a negative marker. In certain embodiments, the marker may code for an antibiotic resistance factor. Suitable examples of antibiotic resistance markers may include, but are not limited to, those coding for proteins that impart resistance to kanamycin, spectinomycin, streptomycin, neomycin, gentamicin (G418), ampicillin, tetracycline, and chloramphenicol. Additionally, the sacB gene may be used as a negative marker. The sacB gene is lethal in many bacteria when they are grown on sucrose media. Additionally, fluorescent proteins may be used as visually identifiable markers. Generally speaking, markers may be present during construction of the strains, but are typically removed from the final constructs. Proteins can also be marked by adding a sequence such as FLAG, HA, His tag, that can be recognized by a monoclonal antibody using immunological methods. In some embodiments, a marker may be a unique identifier of a genetically modified cyanobacterium. In other embodiments, a marker may be a unique identifier of a genetically modified chloroplast genome in a unicellular alga.

B. Spacer Domain

Additionally, a nucleic acid of the invention may comprise a Shine-Dalgarno sequence, or a ribosome binding site (RBS). Generally speaking, a RBS is the nucleic acid sequence in the mRNA that binds to a 16s rRNA in the ribosome to initiate translation. For Gram-negative bacteria, the RBS is generally AGGA. The RBS may be located about 8 to about 11 bp 5′ of the start codon of the first structural gene. One skilled in the art will realize that the RBS sequence or its distance to the start codon may be altered to increase or decrease translation efficiency.

C. Flanking Sequence

Nucleic acid constructs of the invention may also comprise flanking sequences. The phrase “flanking sequence” as used herein, refers to a nucleic acid sequence homologous to a chromosomal sequence. A construct comprising a flanking sequence on either side of a construct (i.e., a left flanking sequence and a right flanking sequence) may homologously recombine with the homologous region of the chromosome, thereby integrating the construct between the flanking sequences into the chromosome. Generally speaking, flanking sequences may be of variable length. In an exemplary embodiment, the flanking sequences may be between about 300 and about 500 bp. In another exemplary embodiment, the left flanking sequence and the right flanking sequence are substantially the same length. For more details, see the Examples.

iv. Plasmids

A nucleic acid construct of the invention may comprise a plasmid suitable for use in a bacterium. Such a plasmid may contain multiple cloning sites for ease in manipulating nucleic acid sequences. Numerous suitable plasmids are known in the art.

### (d) Other Alterations to Facilitate Processing

A recombinant microorganism of the invention may further comprise one or more alterations to further facilitate processing. See for instance WO09155357 and WO11059745, both of which are hereby incorporated by reference in their entirety.

### (e) Alterations to Increase Fatty Acid Secretion

A recombinant microorganism of the invention may further comprise alterations to enable and/or increase fatty acid secretion. See for instance International Publication WO11059745, which is hereby incorporated by reference in its entirety. In one embodiment, a polar cell layer of the microorganism may be altered so as to increase fatty acid secretion. By way of example, the peptidoglycan layer, the outer membrane layer, the S layer of a microorganism, or a combination thereof may be altered to enable increased fatty acid secretion. For instance, expression of a nucleic acid encoding an S-layer protein, such as sll1951, may be decreased or eliminated. In one embodiment, a microorganism is a cyanobacterium, and the microorganism may comprise the mutation Δsll1951.

### (f) Production of High Value Product

The invention may be used to facilitate the production of any high value product that may be synthesized in a microorganism. Non limiting examples of high value products that may be generated in a microorganism may include a biofuel such as biodiesel and jet fuel, a cosmetic, a pharmaceutical agent such as human growth hormone, antibodies and antibody fragments, insulin, tissue plasminogen activator, clotting factor VIII, human lung surfactant, arterial natriuretic hormone, bovine growth hormone, a vaccine such as vaccines for hepatitis B, HPV, hoof-and-mouth disease, and scours, a polysaccharide including cellulose free of lignin, an alcohol, a sugar, a surfactant, a protein, an enzyme such as cellulase, amylase lipase, xylanase, an antimicrobial agent such as penicillin, cephalosporin C, and nisin, a plastic, a vitamin, and a very nutritional easily-digested protein.

In preferred embodiments, the invention may be used to facilitate the production of a fuel. See for instance International Publications WO12003460 and WO11059745, the disclosures of which are hereby incorporated by reference in their entirety. Non-limiting examples of high value products that may be synthesized in phototrophic microorganisms may include biofuels and biofuel precursors, hydrogen gas, and biodegradable plastics such as polyhydroxybutyrate (PHB). In preferred embodiments, high value products that may be synthesized in a phototrophic microorganism may be biofuels and biofuel precursors. Non-limiting examples of biofuels and biofuel precursors that may be produced in a phototrophic microorganism may be free fatty acids, neutral lipids, alkanes, and isoprenoids.

### (g) Preferred Embodiments

In some embodiments, a microorganism is a Synechocystis PCC sp. 6803 cyanobacterium lacking a function encoded by an adhesion protein nucleic acid sequence selected from the group consisting of the protein encoded by the sll1581 nucleic acid sequence of Synechocystis, the protein encoded by the pilC nucleic acid sequence of Synechocystis, the protein encoded by the sll1951 nucleic acid sequence of Synechocystis, the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

In a preferred embodiment, a microorganism is a Synechocystis PCC sp. 6803 cyanobacterium with enhanced adhesion properties, the microorganism comprising a recombinant nucleic acid construct, wherein the nucleic acid construct comprises an inducible promoter operably-linked to a nucleic acid encoding an adhesion protein selected from the group consisting of the protein encoded by the tibA nucleic acid sequence of E. coli, and the protein encoded by the yadA nucleic acid sequence of Yersinia species. In an exemplary embodiment, a microorganism is a Synechocystis PCC sp. 6803 cyanobacterium with enhanced adhesion properties, wherein the microorganism comprises a recombinant nucleic acid construct, wherein the nucleic acid construct comprises an inducible promoter operably-linked to a nucleic acid encoding the protein encoded by the tibA nucleic acid sequence of E. coli as described in the Examples. In another exemplary embodiment, a microorganism is a Synechocystis PCC sp. 6803 cyanobacterium with enhanced adhesion properties, wherein the microorganism comprises a recombinant nucleic acid construct, wherein the nucleic acid construct comprises an inducible promoter operably-linked to a nucleic acid encoding the protein encoded by the yadA nucleic acid sequence of Yersinia species as described in the Examples.

In another preferred embodiment, a microorganism is a Synechocystis PCC sp. 6803 cyanobacterium with enhanced adhesion properties comprising an inactivated adhesion protein, wherein the adhesion protein is selected from the group consisting of the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis. In an exemplary embodiment, a microorganism is a Synechocystis PCC sp. 6803 cyanobacterium with enhanced adhesion properties, comprising an inactivated protein encoded by slr0977 nucleic acid sequence of Synechocystis that has been mutated, deleted, or otherwise altered so as to reduce or eliminate its expression. In another exemplary embodiment, a microorganism is a Synechocystis PCC sp. 6803 cyanobacterium with enhanced adhesion properties comprising an inactivated protein encoded by slr0982 nucleic acid sequence of Synechocystis that has been mutated, deleted, or otherwise altered so as to reduce or eliminate its expression. In yet another exemplary embodiment, the microorganism is a Synechocystis PCC sp. 6803 cyanobacterium with enhanced adhesion properties comprising an inactivated protein encoded by slr1610 nucleic acid sequence of Synechocystis that has been mutated, deleted, or otherwise altered so as to reduce or eliminate its expression.

In yet another preferred embodiment, a microorganism is a Synechocystis PCC sp. 6803 cyanobacterium with defective adhesion properties comprising an inactivated adhesion protein, wherein the adhesion protein is selected from the group consisting of the protein encoded by the sll1581 nucleic acid sequence of Synechocystis, the protein encoded by the pilC nucleic acid sequence of Synechocystis, and the protein encoded by the sll1951 nucleic acid sequence of Synechocystis. In an exemplary embodiment, a microorganism is a Synechocystis PCC sp. 6803 cyanobacterium with defective adhesion properties comprising an inactivated protein encoded by a sll1581 nucleic acid sequence of Synechocystis that has been mutated, deleted, or otherwise altered so as to reduce or eliminate its expression. In another exemplary embodiment, a microorganism is a Synechocystis PCC sp. 6803 cyanobacterium with defective adhesion properties comprising an inactivated protein encoded by a pilC nucleic acid sequence of Synechocystis that has been mutated, deleted, or otherwise altered so as to reduce or eliminate its expression. In yet another exemplary embodiment, a microorganism is a Synechocystis PCC sp. 6803 cyanobacterium with defective adhesion properties comprising an inactivated protein encoded by a eliminatesll1951 nucleic acid sequence of Synechocystis that has been mutated, deleted, or otherwise altered so as to reduce or eliminate its expression.

In some exemplary embodiments, a microorganism is a Synechocystis PCC sp. 6803 cyanobacterium as described in Table 1, Table 4, and Table 5.

### II. Method of Controlling Adhesion

In yet another aspect, the invention comprises a method of controlling adhesion of a phototrophic microorganism. According to the invention, adhesion of a phototrophic microorganism may be increased or decreased to control, for example, autoagglutination of the microorganism, and biofilm formation.

In some embodiments, a method of the invention comprises introducing into the microorganism a recombinant nucleic acid construct, wherein the nucleic acid construct comprises an inducible promoter operably-linked to a nucleic acid encoding an adhesion protein, wherein the microorganism is capable of regulated expression of the nucleic acid encoding the adhesion protein. According to the invention, adhesion of a microorganism capable of regulated expression of a nucleic acid encoding an adhesion protein may be controlled by expressing the nucleic acid encoding the adhesion protein in the microorganism to enhance the adhesion of the microorganism, by reducing the expression of the nucleic acid encoding the adhesion protein to reduce the adhesion of the microorganism, or by reducing the expression of the nucleic acid encoding the adhesion protein to enhance the adhesion of the microorganism.

In one embodiment, adhesion of a microorganism capable of regulated expression of a nucleic acid encoding an adhesion protein may be controlled by expressing the nucleic acid encoding the adhesion protein in the microorganism to enhance the adhesion of the microorganism. In another embodiment, adhesion of a microorganism capable of regulated expression of a nucleic acid encoding an adhesion protein may be controlled by reducing the expression of the nucleic acid encoding the adhesion protein to reduce the adhesion of the microorganism. In yet another embodiment, adhesion of a microorganism capable of regulated expression of a nucleic acid encoding an adhesion protein may be controlled by reducing the expression of the nucleic acid encoding the adhesion protein to enhance the adhesion of the microorganism.

In other embodiments, a method of the invention comprises reducing the adhesion of a phototrophic microorganism by removing from the microorganism an adhesion function encoded by a nucleic acid encoding an adhesion protein selected from the group consisting of the protein encoded by the sll1581 nucleic acid sequence of Synechocystis, the protein encoded by the pilC nucleic acid sequence of Synechocystis, and the protein encoded by the sll1951 nucleic acid sequence of Synechocystis.

In yet other embodiments, a method of the invention comprises enhancing the adhesion of a phototrophic microorganism by removing from the microorganism an adhesion function of an adhesion protein selected from the group consisting of the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

Phototrophic microorganisms, adhesion proteins, nucleic acid constructs, and methods of regulating synthesis of an adhesion protein in a microorganism of the invention are as described in Section I. In preferred embodiments, the phototrophic microorganism is a Synechocystis PCC sp. 6803 cyanobacterium.

### III. Method for Harvesting a Microorganism

Another aspect of the present invention is a method for harvesting a microorganism. Microbial harvesting and biomass dewatering of a culture of microorganisms is the most energy intensive step in large-scale production of industrially valuable products in microorganisms. For instance, harvesting microbial biomass may include centrifugation, filtration or flocculation using chemical additives that may be costly and/or toxic. Autoagglutination, or clumping together of cells of the microorganism of the invention, may lead to the settling of the autoagglutinated clumps, and may facilitate biomass collection of the microorganism of the invention in the absence of energy intensive centrifugation, filtration or the use of chemical additives. As such, autoagglutination facilitates harvesting the biomass for extraction and processing of cell contents from the microorganism. Such cell contents may include biofuels and other industrially valuable products that may be produced in the microorganisms, or discarded biomass that may be used in agricultural animal feeds and fertilizers.

Generally speaking, a method of the invention comprises introducing into a microorganism a nucleic acid comprising an inducible promoter operably-linked to a nucleic acid encoding an adhesion protein capable of inducing adhesion of a microorganism when synthesized in the microorganism. Adhesion proteins capable of inducing adhesion of the microorganism may be as described in Section I(b). Methods of introducing a nucleic acid comprising an inducible promoter operably-linked to a nucleic acid encoding an adhesion protein are described in Section I above. In preferred embodiments, the microorganism is a Synechocystis phototrophic microorganism.

In an exemplary embodiment, the microorganism is the SD342 strain of Synechocystis described in the Examples, wherein the Synechocystis strain comprises a nucleic acid construct, wherein the nucleic acid construct comprises an inducible promoter induced by the lack of CO₂ operably-linked to a nucleic acid encoding an adhesion protein encoded by the tibA nucleic acid sequence of Yersinia species. In another exemplary embodiment, the microorganism is the SD1014 strain of Synechocystis described in the Examples, wherein the Synechocystis strain comprises a nucleic acid construct, wherein the nucleic acid construct comprises an inducible promoter induced by the P_{nrsB} Ni metal, operably-linked to a nucleic acid encoding an adhesion protein encoded by the yadA nucleic acid sequence of Yersinia species. In yet another exemplary embodiment, the microorganism is the SD1014 strain of Synechocystis described in the Examples, wherein the Synechocystis strain comprises a nucleic acid construct, wherein the nucleic acid construct comprises an inducible promoter induced by the P_{nrsB} Ni metal, operably-linked to a nucleic acid encoding an adhesion protein encoded by the aipA nucleic acid sequence of Proteus mirabilis. In another exemplary embodiment, the microorganism is the SD1014 strain of Synechocystis described in the Examples, wherein the Synechocystis strain comprises a nucleic acid construct, wherein the nucleic acid construct comprises an inducible promoter induced by the P_{nrsB} Ni metal, operably-linked to a nucleic acid encoding an adhesion protein encoded by the tibA nucleic acid sequence of E. coli. In still another exemplary embodiment, the microorganism is the SD1014 strain of Synechocystis described in the Examples, wherein the Synechocystis strain comprises a nucleic acid construct, wherein the nucleic acid construct comprises an inducible promoter induced by the P_{nrsB} Ni metal, operably-linked to a nucleic acid encoding an adhesion protein encoded by the taaP nucleic acid sequence of Proteus mirabilis.

A microorganism comprising an inducible promoter operably-linked to a nucleic acid encoding an adhesion protein is cultured. Methods of culturing a microorganism are known in the art and detailed in the examples.

A method of the invention further comprises inducing a promoter to cause synthesis of an adhesion protein for agglutination. For instance, when a promoter is a promoter induced by the lack of CO₂, the promoter may be induced by sealing the culture to initiate carbon dioxide limitation. Alternatively, when a promoter is a promoter induced by Ni, the promoter may be induced by the addition of Ni to the culture medium. In some embodiments, Ni may be added to about 0.5, 1, 1.5, 2, 2.5, 3, 3.5, 4, 4.5, 5, 5.5, 6, 6.5, 7, 7.5, 8, 8.5, 9, 9.5, or about 10 μM to induce the promoter. In a preferred embodiment, Ni may be added to about 1, 1.5, 2, 2.5, or about 3 μM to induce the promoter. In another preferred embodiment, Ni may be added to about 5, 5.5, 6, 6.5, or about 7 μM to induce the promoter.

After induction of a promoter to cause synthesis of an adhesion protein, a microorganism may then be allowed to autoagglutinate before collecting the autoagglutinated biomass. The duration for allowing microorganisms to agglutinate can and will vary depending on the culture conditions, the strain of the microorganism, the protein capable of inducing autoagglutination, the level of expression of the adhesion protein, culture conditions, and other variables, and may be determined by experimentation. The duration for allowing the microorganisms to agglutinate may also be controlled. For instance, the duration for allowing microorganisms to agglutinate may be decreased by increasing the synthesis level of an adhesion protein, by inactivation of an adhesion protein by the expression of more than one nucleic acid sequence encoding an adhesion protein, or by synthesizing a secondary gene product to accelerate the autoagglutination capabilities of the microorganism.

In some embodiments, microorganisms may be allowed to autoagglutinate for about 6, 12, 24, 30, 36, 42, 48, 54, 60, 66, or about 72 hours. In other embodiments, microorganisms may be allowed to autoagglutinate for about 1, 2, 3, 4, 5, 6, 7, 8, 9, or about 10 days. In some embodiments, microorganisms may be allowed to autoagglutinate for about 35, 36, 37, 38, 39, 40, 41, 42, 43, 44, 45, 46, 47, 48, 49, 50, 51, 52, 53, 54, 55, 56, 57, 58, 59 or about 60 hours. In preferred embodiments, microorganisms may be allowed to autoagglutinate for about 45, 46, 47, 48, 49, 50, or about 51 hours.

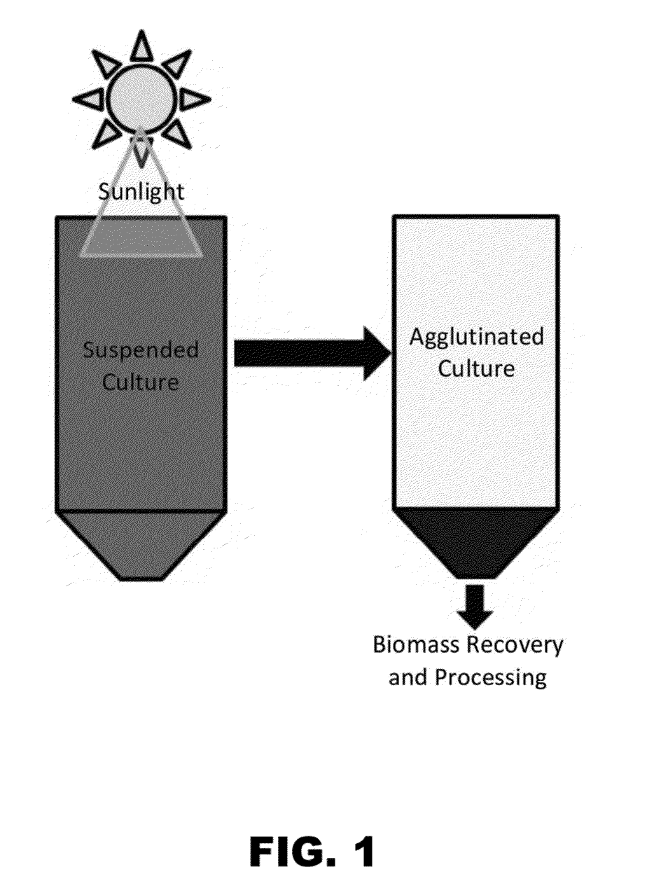

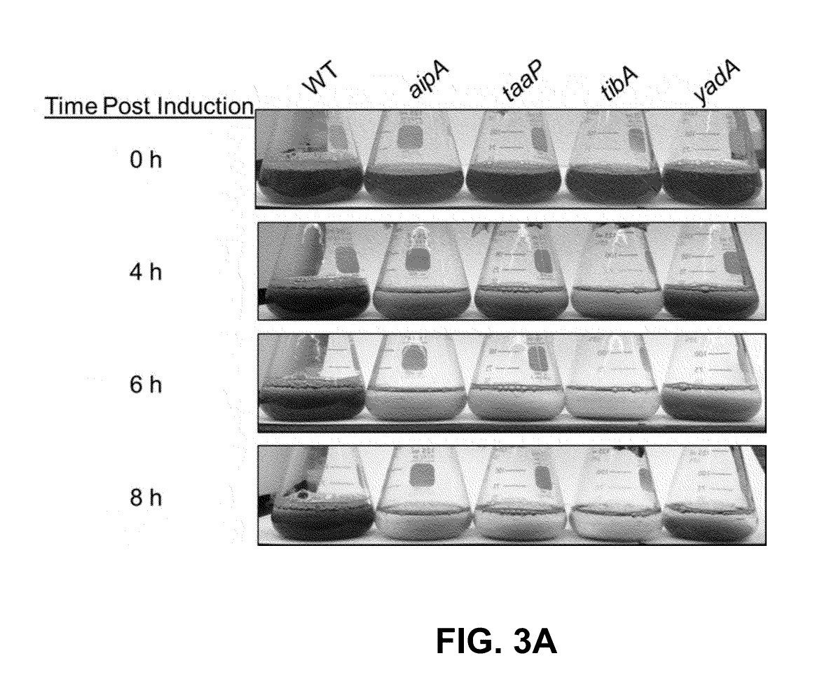

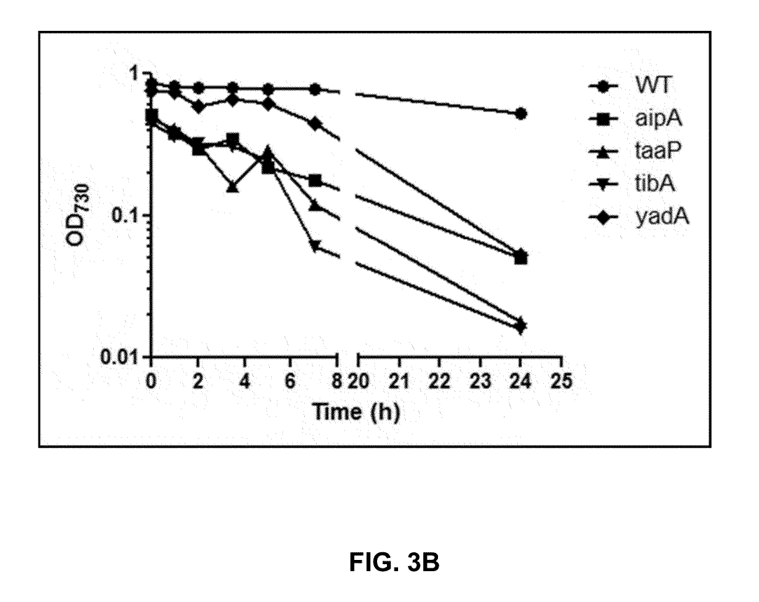

Autoagglutinated microorganisms are denser than free floating microorganisms that are not autoagglutinated and may then settle as an autoagglutinated mass at the bottom of the culture vessel as described in FIG. 1 and FIG. 3. The autoagglutinated biomass may then be collected for further processing.

In some embodiments, when an inducible promoter is induced by the lack of CO₂, the promoter may be induced by confining the culture comprising a microorganism in sealed vessels for CO₂ limitation. The duration of CO₂ limitation can and will vary depending on the culture conditions, the strain of the microorganism, the level of adhesion protein synthesis, and other variables, and may be determined by experimentation as described in the Examples below. In some embodiments, a promoter may be induced by the lack of CO₂ for about 6, 12, 24, 30, 36, 42, 48, 54, 60, 66, or 72 hours. In other embodiments, the promoter may be induced by the lack of CO₂ for about 1, 2, 3, 4, 5, 6, 7, 8, 9, or 10 days. In preferred embodiments, a promoter may be induced by the lack of CO₂ for about 2 days.

In some embodiments, methods of the invention may be used to facilitate harvesting of biofuels and biofuel precursors produced by a microorganism of the invention. Biofuels and biofuel precursors produced by a microorganism may be extracted from a culture media or culture biomass. Methods of extracting fatty acids from phototrophic microorganism cultures are known in the art. For instance, a fatty acids biofuel may be pipetted, filtered, skimmed from the culture media, and/or the fatty acids may be recovered by the use of resins that bind the fatty acids. Such extractions by binding to resins may be conducted continuously during growth of a microorganism producing fatty acids. In addition, a culture media may be treated to extract any remaining fatty acids dissolved in the media. Briefly, media may be acidified and extracted with an organic solvent, such as hexane. The organic phase may then be separated and dried to isolate the fatty acids. In some embodiments, media is extracted more than once with the organic solvent. For instance, media may be extracted two, three, four or five times.

### IV. Method of Controlling Biofilm Formation

In yet another aspect, the invention comprises a method of controlling biofilm formation. As used herein, the term “biofilm” may refer to an aggregate of microorganisms in which cells adhere to a solid surface and to each other to form a film. These adherent cells may be embedded within a self-produced matrix of extracellular polymeric substance generally composed of extracellular DNA, proteins, and polysaccharides. According to the invention, biofilm formation may be controlled by regulating the synthesis of an adhesion protein in the microorganism. Adhesion proteins and methods of regulating synthesis of an adhesion protein in a microorganism of the invention are as described in Section I.

### (a) Preventing Biofilm Formation

In some embodiments, a method may be used to prevent biofilm formation. For instance, a method may be used to prevent biofilm formation in a bioreactor to prevent biofouling of the bioreactor, which may decrease efficiency of the bioreactor. As used herein, the term “bioreactor” may refer to any container and equipment in contact with a culture of microorganisms that may be used for culturing a microorganism, and may include small scale culture containers and equipment normally used in a laboratory setting, larger scale bioreactors normally used in industrial production of valuable products using microorganisms, or tanks used for bioremediation.

Generally speaking, biofilm formation may be prevented by inactivating or regulating synthesis of one or more adhesion proteins. Adhesion proteins capable of inducing adhesion when synthesized in a microorganism may be as described in Section I(b). Altering the expression of a nucleic acid sequence encoding an adhesion protein may be as described in Section I(c).

In preferred embodiments, a microorganism is Synechocystis, and biofilm formation may be prevented by reducing synthesis of an adhesion protein. In an exemplary embodiment, a microorganism is Synechocystis, and biofilm formation may be prevented in Synechocystis by reducing expression of the sll1581 nucleic acid sequence of Synechocystis. In another exemplary embodiment, the microorganism is Synechocystis, and biofilm formation may be prevented in Synechocystis by reducing expression of the pilC nucleic acid sequence of Synechocystis. In yet another exemplary embodiment, the microorganism is Synechocystis, and biofilm formation may be prevented in Synechocystis by reducing the expression of the sll1951 nucleic acid sequence of Synechocystis. Biofilm formation may be prevented to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or about 40%, more preferably to about 15, 16, 17, 18, 19, 20, 21, 22, 23, 24, or about 25% of the level of biofilm formation of a microorganism comprising a wild type adhesion protein.

### (b) Inducing Biofilm Formation

In other embodiments, a method may be used to induce biofilm formation. For instance, a method may be used to generate an immobilized cell bioreactor. As used herein, the term “immobilized cell bioreactor” may refer to any bioreactor wherein the cells are not suspended in the culture media, but immobilized on a solid support. Non-limiting examples of immobilized cell bioreactors may include moving media, or Moving Bed Biofilm Reactor (MBBR), packed bed bioreactors, fibrous bed bioreactors, and membrane bioreactors. Using such bioreactors may increase the efficiency of the bioreactor by providing higher biomass density in the bioreactor, increased production rates, and may allow for reuse of microorganisms in the bioreactor without the need of re-inoculation in repeat batch fermentation.

In one embodiment, biofilm formation may be induced by inactivating or regulating the synthesis of one or more adhesion proteins in a microorganism. In a preferred embodiment, biofilm formation may be induced by inactivating or preventing the synthesis of one or more adhesion proteins.

Adhesion proteins that, when inactivated in a microorganism may lead to enhanced adhesion of the microorganism, are as described in Section I(b). Adhesion proteins capable of inducing adhesion when synthesized in a microorganism may be as described in Section I(b). Inactivating synthesis of an adhesion protein may be as described in Section I(c).

In preferred embodiments, a microorganism is Synechocystis, and biofilm formation may be enhanced by inactivating an adhesion protein. In an exemplary embodiment, a microorganism is Synechocystis, and biofilm formation may be enhanced by inactivating the adhesion protein encoded by the slr0977 nucleic acid sequence of Synechocystis, which putatively encodes a permease component of an ABC transporter. In another exemplary embodiment, a microorganism is Synechocystis, and biofilm formation may be enhanced by inactivating the adhesion protein encoded by the slr0982 nucleic acid sequence of Synechocystis, which putatively encodes a polysaccharide ABC transporter ATP binding subunit. In yet another exemplary embodiment, a microorganism is Synechocystis, and biofilm formation may be enhanced by inactivating the adhesion protein encoded by the slr1610 nucleic acid sequence of Synechocystis, which putatively encodes a C-3 methyl transferase. Biofilm formation may be enhanced by about 0.5, 1, 1.5, 2, 2.5, 3, 3.5, 4 fold or more compared to the level of biofilm formation of a microorganism comprising a wild-type adhesion protein.

Methods of measuring biofilm formation are known in the art and may include Quartz crystal microbalance (QCM), microjet impingement (MJI), direct enumeration of bacteria in biofilms, the air-liquid interface (ALI) assay, the colony biofilm assay, the kadouri drip-fed biofilm assay, and the crystal violet assay. In preferred embodiments, biofilm formation is measured using the crystal violet assay as described in the examples.

### DEFINITIONS

The term “cell wall”, as used herein, refers to the peptidoglycan layer of the cell wall of a cyanobacterium, or the cell wall of algal cells comprising polysaccharides and glycoproteins. Stated another way, “cell wall” as used herein refers to the rigid layer of the cell wall.

The term “operably-linked”, as used herein, means that expression of a nucleic acid sequence is under the control of a promoter with which it is spatially connected. A promoter may be positioned 5′ (upstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “promoter”, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. In some embodiments, activators may bind to promoters 5′ of the -35 RNA polymerase recognition sequence, and repressors may bind 3′ to the -10 ribosome binding sequence.

The term “adhesion protein” as used herein, refers to any protein that may alter the adhesive properties of a microorganism when the synthesis of the adhesion protein in the microorganism is altered.

The terms “defective adhesion”, “reduced adhesion”, “decreased adhesion”, and “non-biofouling” may be used interchangeably and may refer to lower levels of adhesion of a microorganism comprising altered synthesis of adhesion protein, when compared to the levels of adhesion of the microorganism when the synthesis of the adhesion protein is not altered.

The terms “enhanced adhesion”, “improved adhesion”, and “biofilm formation” refer to higher levels of adhesion of the microorganism comprising the altered synthesis of an adhesion protein, when compared to the levels of adhesion of the microorganism when the synthesis of the adhesion protein is not altered.

### EXAMPLES

The following examples illustrate various iterations of the invention.

### Example 1

### Carbon Dioxide-Induced Autoagglutination of Synechocystis

Biomass harvesting and dewatering is the most energy intensive step in large-scale biofuel production with photosynthetic microorganisms and include centrifugation, filtration, or flocculation by chemical additives that may be costly and/or toxic. The inventors devised an efficient, inexpensive method for biomass recovery. In short, the inventors generated a photosynthetic microorganism with controlled induction of an E. coli gene encoding an adhesion protein for the dewatering of biomass through the genetic control of autoaggregation (FIG. 1).

The tibA gene from E. coli, which encodes a Type V secreted adhesin, was amplified from E. coli (ATCC 35401) genomic DNA by using the Polymerase Chain Reaction (PCR), as was the kanamycin-resistance gene (amplified from pPsbA2kS). Similarly, the cmpR and cmpA genes were amplified by PCR from Synechocystis PCC 6803 genomic DNA. The cmpR gene encodes a transcriptional regulator that activates CO₂-dependent expression of the bicarbonate transporter encoded by cmpA gene. These three PCR products were then ligated together and amplified by PCR such that the final amplicon contained cmpR, tibA, Kan^{R} and cmpA in that order. In this way, the genes encoding TibA and the kanamycin-resistance marker were placed under the control of P_{cmpA}. The final amplicon was then cloned into pJet1.2 (Fermentas) to generate pψ342, which acts as a suicide plasmid in Synechocystis PCC 6803.

More specifically, to generate pψ342, tibA was first fused to a kanamycin-resistance marker. This PCR fusion was then cloned into pψ234 to place tibA::KanR under control of P_{cpmA} [8]. To this end, PCR primers TibA-S-S6F1 and S6F1-A-TibA (Table 2) were used to amplify tibA from E. coli genomic DNA (ATCC H10407). The kanamycin-resistance marker was amplified from pPsbA2ks using primers TibA-A-KM and KM-S-TibA (Table 2). The tibA and KanR amplicons were then fused using sewing PCR as previously described (Warren 1997). pψ234 was amplified with primers cmpA-s and cmpR-a (Table 2). The tibA::KanR amplicon was phosphorylated with T4 phosphonucleotide kinase (New England Biolabs) according to manufacturer's instructions. These two PCR products were ligated overnight and transformed into TOP10 E. coli. Transformants were selected on LB plates containing 50 μg/ml kanamycin (Sigma).

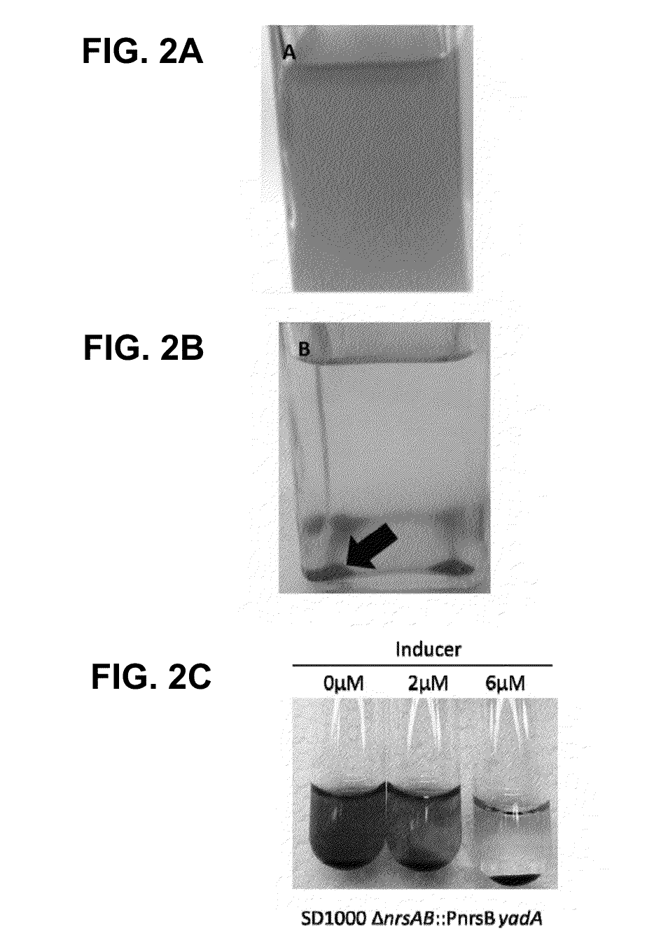

Wild-type Synechocystis cells (strain SD100) were naturally transformed with pψ342 plasmid DNA. The transformation reaction was then plated on a 0.4 μm filter placed on a BG-11 plate. After incubation overnight at 30° C. with 50 μmol photons m⁻² sec⁻¹, the transformants were transferred to a BG-11 plate with 50 μg kanamycin/ml. Kanamycin-resistant colonies were tested by PCR for the presence of tibA. Colonies that were positive for tibA were tested for autoagglutination by growing cells to late log phase at which point cultures were sealed to initiate carbon dioxide limitation and expression from the P_{cmpA} promoter which resulted in autoagglutination and dewatering of the microbial biomass (FIGS. 2A and B).

### Example 2

### NiCl₂-Induced Autoagglutiantion of Synechocystis

A Synechocystis strain (SD1014) was also constructed with the yadA nucleic acid sequence of Yersinia species encoding an adhesion protein operably linked to the Ni-induced promoter P_{nrsB} as described above.

SD1000 was naturally transformed with pψ637. The transformation reaction was then plated on a 0.4 μm filter placed on a BG-11 plate. After incubation overnight at 30° C. with 50 μmol photons m⁻² sec⁻¹, the 0.4 μm filter containing the transformants was transferred to a BG-11 plate with 50 μg kanamycin/ml. Kanamycin-resistant colonies were tested by PCR for the presence of yadA and tested for autoagglutination to generate SD1014.

Autoagglutination of the SD1014 strain was induced using various concentrations of Ni (FIG. 2C). Specifically, three 125 mL cultures of SD1014 in BG-11+0.1 M NaCl were inoculated to an OD₇₃₀ of 0.06 and grown as described above to an OD₇₃₀ of 0.355. Each culture received 0, 2 μM, or 6 μM NiCl₂ and were grown to stationary phase with bubbling. Cells were then grown to stationary phase. To test for autoagglutination, 2 mL of culture were then removed from the flask and allowed to settle for 48 hours, resulting in complete precipitation of biomass (FIG. 2C).

### Example 3

### NiCl₂-Induced Autoagglutintion of Synechocystis Using Adhesins Encoded by aipA, taaP, tibA, or yadA

Synechocystis strains expressing adhesins encoded by aipA, taaP, or tibA under the control of the Ni-induced promoter P_{nrsB} were generated as described for the Synechocystis strain SD1014 in Example 2.

Autoagglutination of a wild-type Synechocystis, the Synechocystis strain SD1014, and the Synechocystis strains synthesizing adhesins encoded by aipA, taaP, or tibA, was tested as follows. Strains were grown in 20 ml of BG-11 at 30° C. with 50 μmol photons/sec/m² to and OD₇₃₀ of ˜0.6. All cultures were then induced with 6 μM NiCl₂ for 48 hours, and allowed to settle for about 4, 6, or about 8 hours (FIG. 3A).

Autoagglutination and precipitation from the culture medium of wild-type Synechocystis, Synechocystis strain SD1014, and the Synechocystis strains synthesizing adhesins encoded by aipA, taaP, or tibA, were quantified by measuring the optical density (OD₇₃₀) of a 1 ml sample removed from the top 1 cm of each culture at various time points (FIG. 3B).

### Example 4

### Engineering a Non-Biofouling Strain of Synechocystis

Genes of interest for biofilm formation/adhesion in Synechocystis were identified by sequence homology search of the annotated Synechocystis genome for genes known to contribute to biofilm formation in Caulobacter crescentus, Escherichia coli, and Salmonella typhimurium, among others. Additionally, genes identified in the Synechocystis literature as coding for cell surface molecules such as S-layer (Sakiyama 2006) and pili (Yoshihara 2004) were also targeted for characterization. Genes of interest were knocked out using double-homologous recombination of DNA in vivo. Formation of biofilm by wild-type and mutant strains of Synechocystis was characterized.

Representative mutants and their phenotypes are shown in Table 5. Substituting native promoters with inducible promoters such as a CO₂-limitation inducible promoter, stationary-phase inducible promoter, or other promoters may enable overexpression or temporal modulation of cell surface molecules shown to contribute to adhesion/biofilm formation.

Biofilm formation was assessed by adapting the crystal violet assay, which is a standard assay used to measure biofilm formation in laboratory cultures. Specifically, cells that attached to a surface are stained with crystal violet, which binds to the negatively charged cell wall. Unbound crystal violet is rinsed off, and bound stain is eluted with DMSO and measured by quantifying absorbance at 600 nm. Higher crystal violet absorbance at 600 nm corresponds to more biofilm formation.

The crystal violet assay for measurement of biofilm formation by a phototrophic microbe is performed as described previously (Bodenmiller et al. 2004 JBAC), with modifications. The starter culture for assessment of biofilm formation is grown under nutrient replete conditions (1×BG-11), and shifted to nutrient deplete conditions within the 12-well plate (0.85×BG-11). The plate is sealed with parafilm and transparent adhesive tape and incubated with 32 uMol photons PAR/m2/sec at 71 rpm shaking for 72 hours.

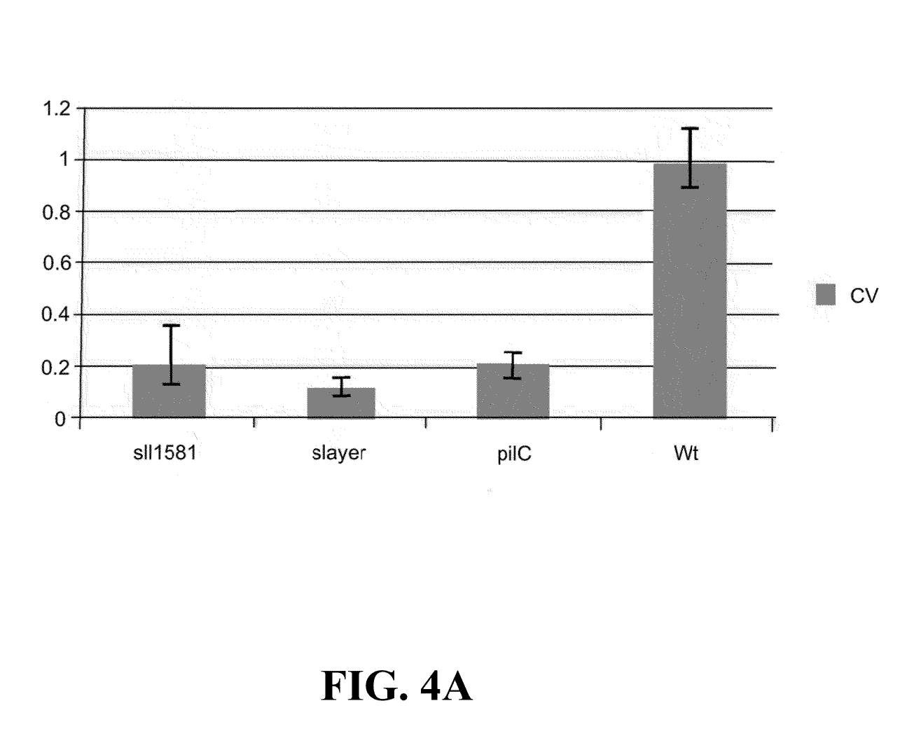

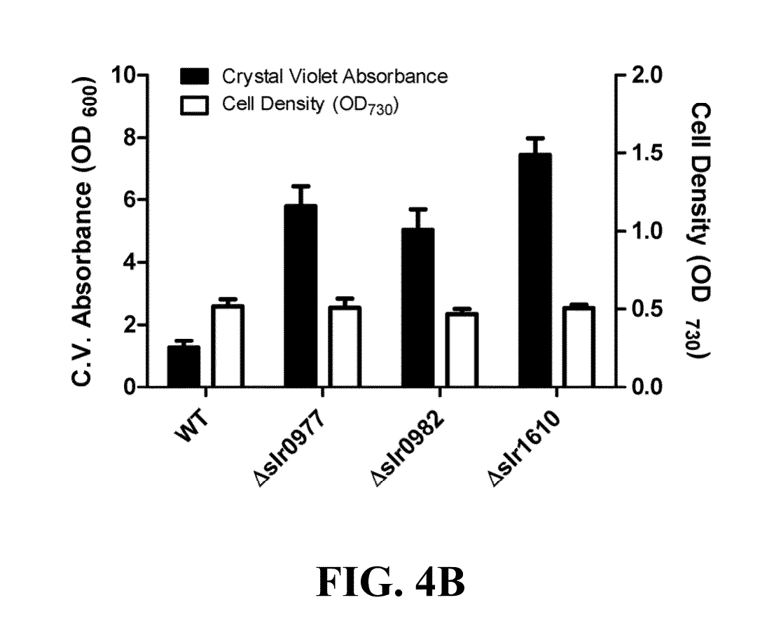

All mutant strains of Synechocystis tested showed reduced levels of biofilm formation compared to wild-type control Synechocystis (FIG. 4A).

### Example 5

### Engineering a Strain of Synechocystis with Enhanced Biofilm Formation

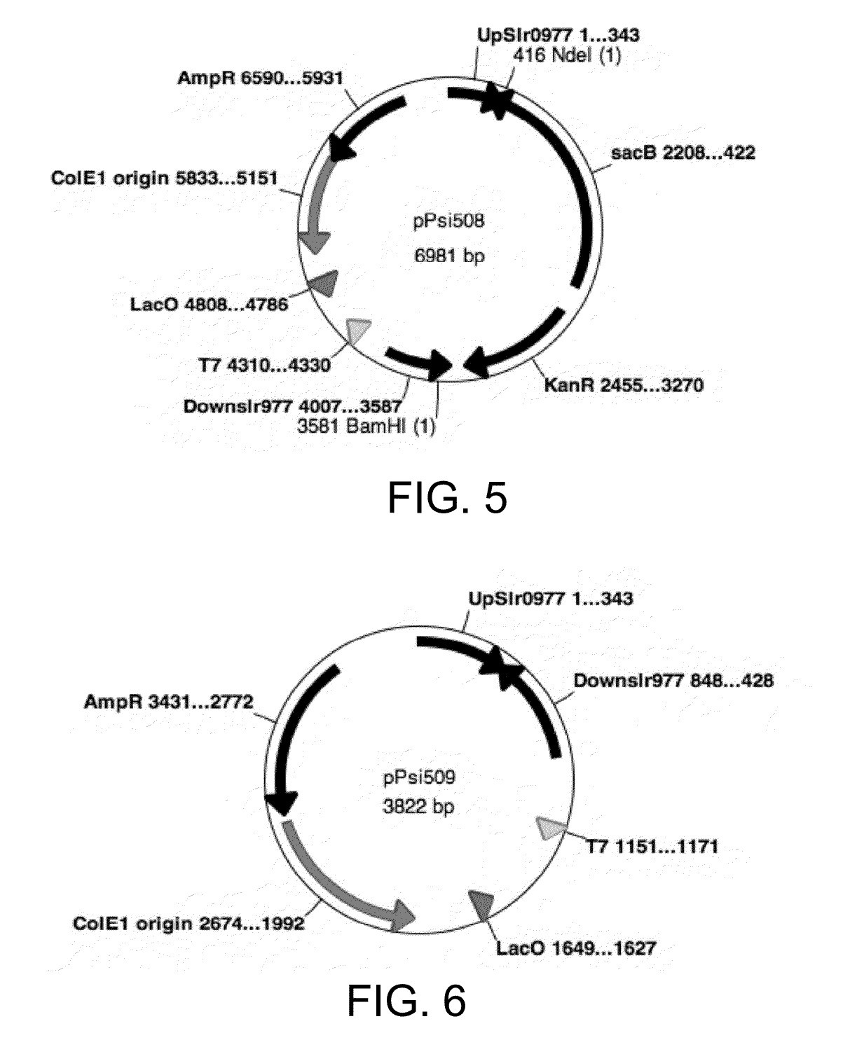

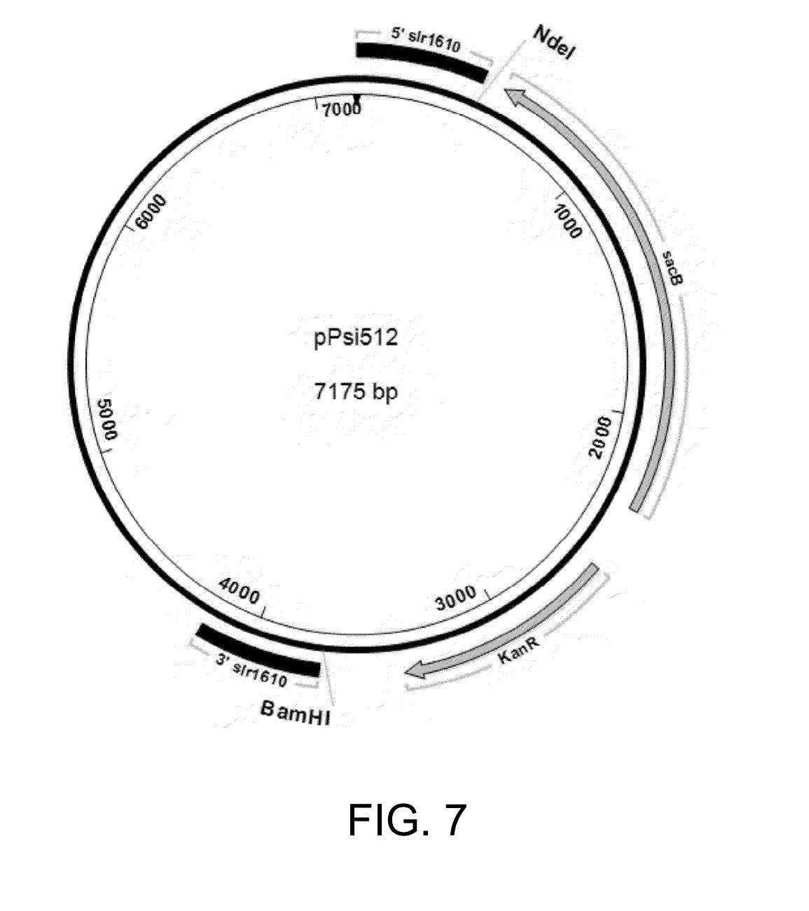

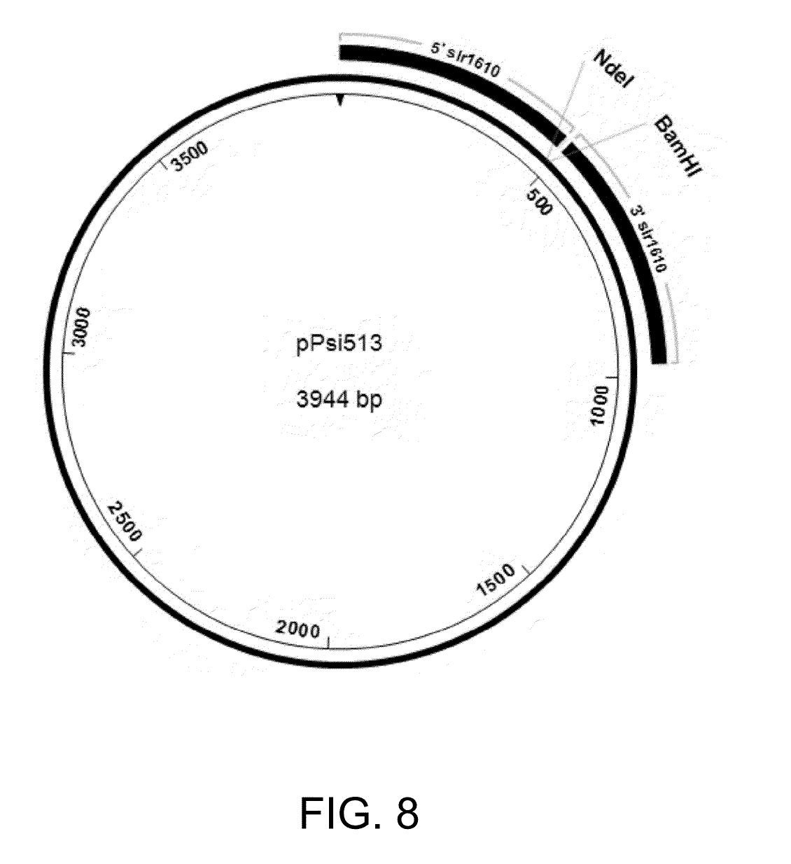

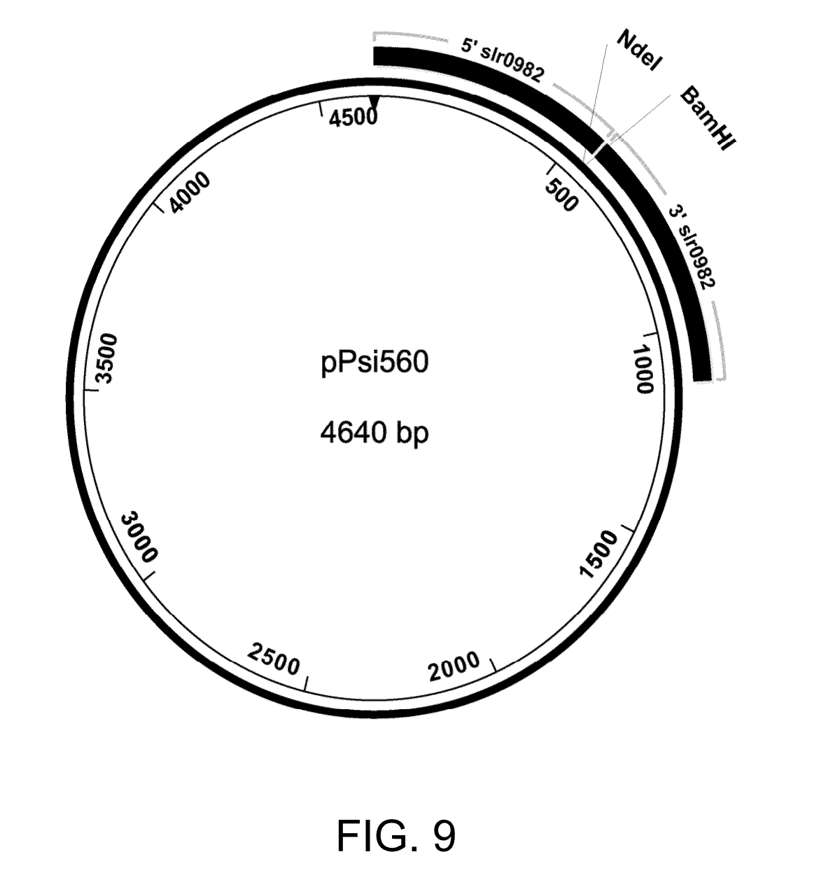

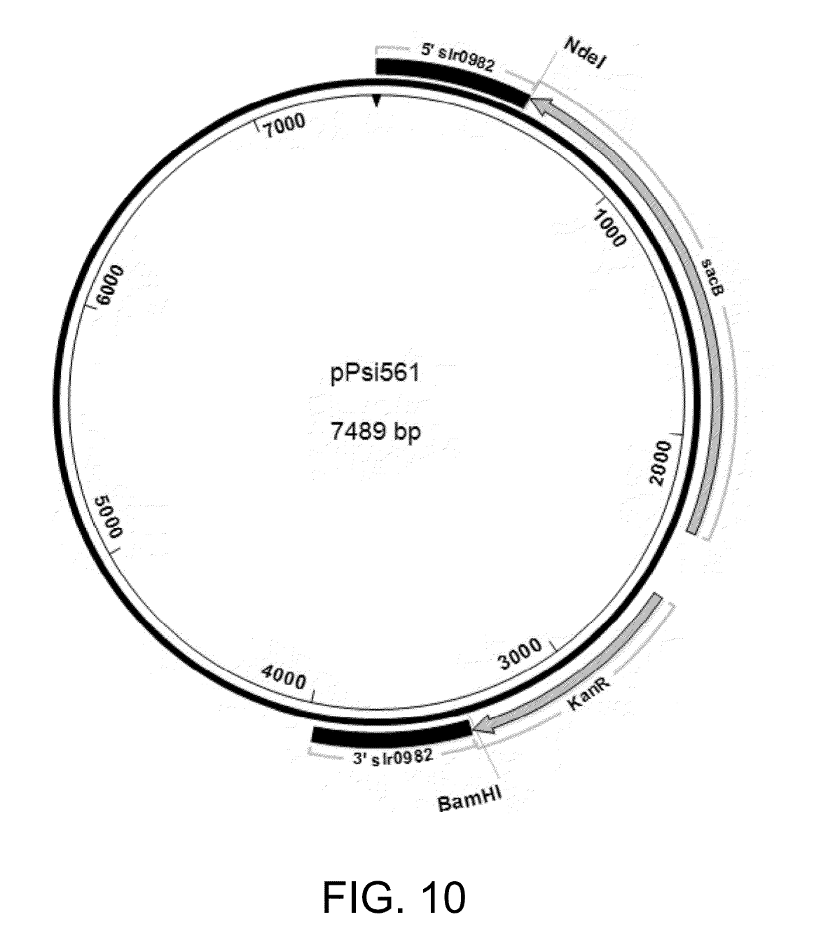

Genes with potential involvement in cell surface modification were identified by bioinformatic analysis. The genes slr0977, slr0982 and slr1610 are part of an operon in Synechocystis that function in the export of extracellular polysaccharides (EPS). slr0977 encodes a membrane permease through which EPS is delivered to the cell membrane. slr0982 encodes an ATP-binding cassette protein, which is believed to provide the energy necessary for EPS transport through the permease encoded by slr0977. slr1610 is a putative methyltransferase that functions in chain termination of EPS elongation. Synechocystis genes slr0977, slr0982, or slr1610 were knocked out using double-homologous recombination of DNA in vivo. DNA manipulation was carried out using standard procedures. To construct suicide vectors pψ509 (FIG. 6), pψ513 (FIG. 8), and pψ561 (FIG. 10), PCR primers (Table 2) were used to amplify genomic DNA from the flanking region of each gene to be disrupted. These flanking regions were stitched together by sewing PCR as previously described (Warren 1997) such that BamHI and NdeI restriction sites were generated between the two flanking sequences. In the case of pψ561, an NdeI site native to the 3′ flanking region was removed by introducing a silent mutation using QuikChange II Site-Directed Mutagenesis Kit (Agilent Technologies) according to the manufacturer's protocol, using primers MLF-68 and MLF-69. The Kan^{R}-SacB cassette from pPbs2ks was purified following digestion with BamHI and NdeI and ligated into pψ509 (FIG. 6), pψ513 (FIG. 8) and pψ561 (FIG. 10) to generate pψ508 (FIG. 5), pψ512 (FIG. 7) and pψ560 (FIG. 9), respectively.

Adherence by wild-type and mutant strains of Synechocystis was characterized, and showed significantly increased adherence in knockout Synechocystis strains compared to wild-type Synechocystis strains (FIG. 4B). Synechocystis cultures were inoculated at an OD₇₃₀ of 0.1 in two mL of BG-11 medium in test tubes. Kanamycin was added at a concentration of 50 μg/mL when appropriate. In each experimental replicate, quintuplicate cultures were grown without shaking for 96 hours at 30° C. with 40 μmol photons m⁻¹ s⁻¹. The culture medium containing non-adherent cells was decanted from each of the tubes. Each tube was gently washed 2× with 2 mL of BG-11. Tubes were then stained with 1% crystal violet in _{dd}H₂O for 15 min. The tubes were then gently rinsed 3× with _{dd}H₂O. Adherent cells were resuspended in 1 mL of DMSO and vortexed vigorously. The OD₆₀₀ of each tube was then measured. To control for differences in growth rate between strains, the remaining two culture tubes were vortexed vigorously for 15 seconds or until adhered cells were fully in suspension. The OD₇₃₀ of these cultures was then measured and the average value was calculated to include all experiments.

### Example 6

### Other Plasmids and Strains

A series of plasmids were constructed to place the nucleic acid encoding for adhesins under the control of P_{nrsB}. The 5′ and 3′ flanking regions of P_{nrsB} were amplified using primers MLF-117, MLF-112, MLF-96, and MLF-97 (Table 2). The resulting two PCR products were fused using sewing PCR as described above such that the fusion amplicon containing BamHI and SacI sites was introduced between the 5′ and 3′ flanking regions of P_{nrsB}. To generate pψ588 this fused PCR product was ligated into pJet1.2.

The aipA gene was amplified from pSAP2022 [6] using primers MLF-173 and MLF174. The Kan^{R} cassette was PCR amplified from pψ511 using primers MLF-175 and MLF176. These two PCR fragments were fused as above such that an SphI site was introduced between aipA and the KanR cassette. This fused amplicon was then cloned into pψ588 following digestion with BamHI and SacI to generate pψ630. The genes taaP and yadA were amplified using primers pairs MLF-185/MLF-172 and MLF-181/MLF-182, respectively. pSAP2022 was used as the template for aipA. pSAP2025 was used as the template for taaP [6] The taaP PCR product was cloned into the BamHI-SphI sites of pψ630 to generate pψ635. The yadA PCR product was cloned into the BamHI-SphI sites of pψ630 to generate pψ637.

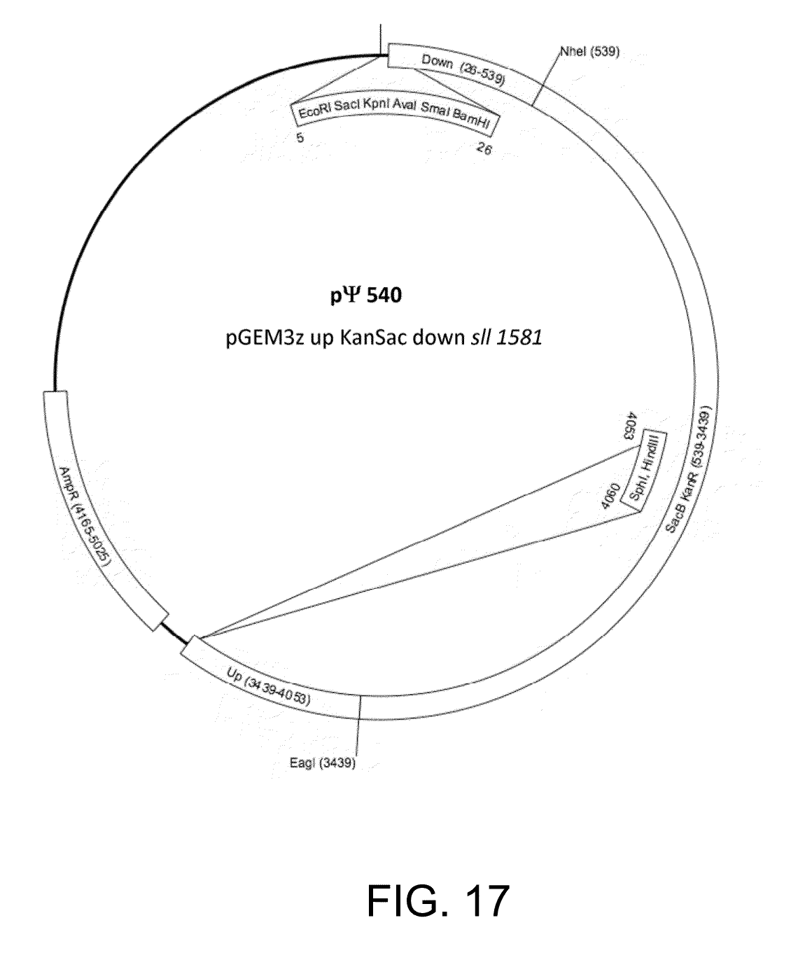

To construct suicide vectors pψ540, ψ541, and ψ543, PCR primers (Table 2) were used to amplify genomic DNA from the flanking region of each gene to be disrupted. Regions upstream and downstream were cloned into the commercial vector pGEM 3z (Promega) using the BamHI and SphI restriction sites. The KanR-sacB fragment was amplified from the pPbs2ks plasmid and inserted between flanking regions using NheI and EagI sites in a four-part ligation reaction (FIG. 17).

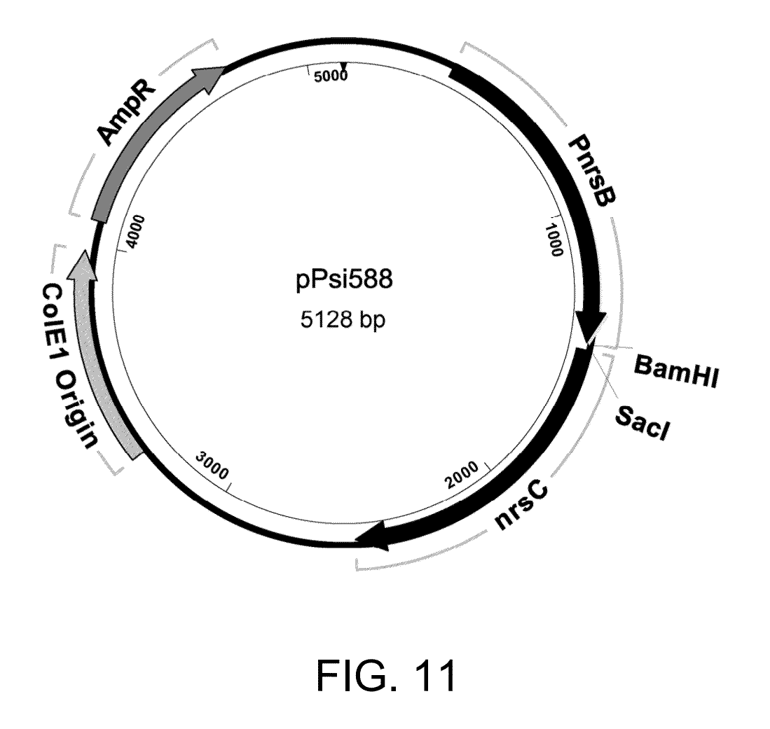

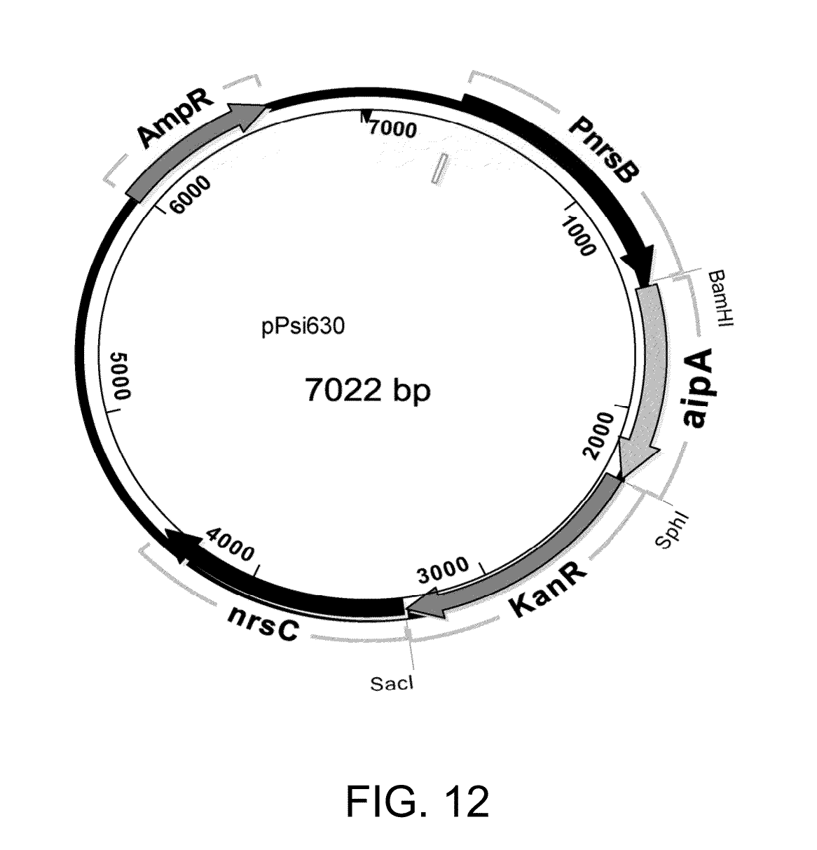

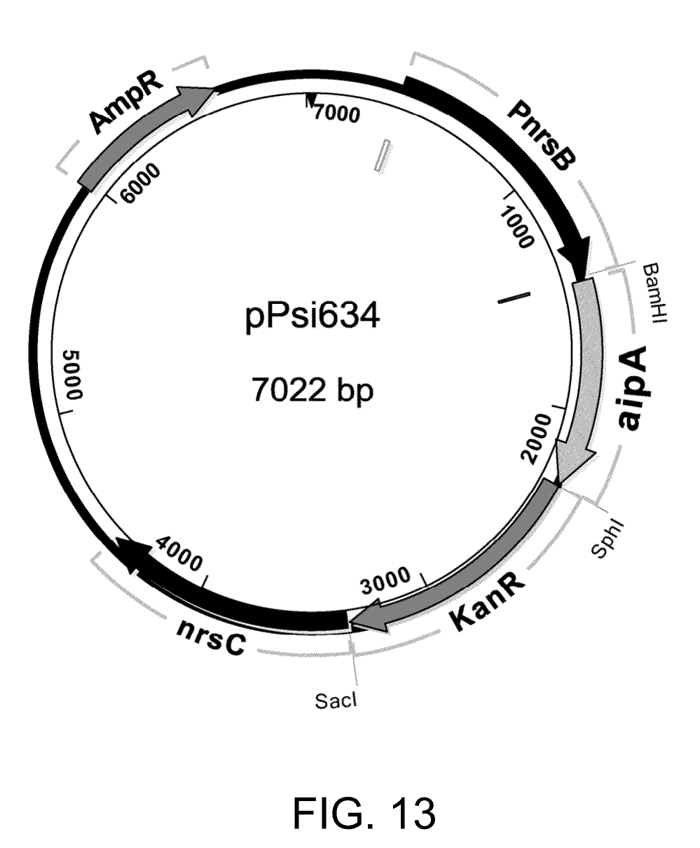

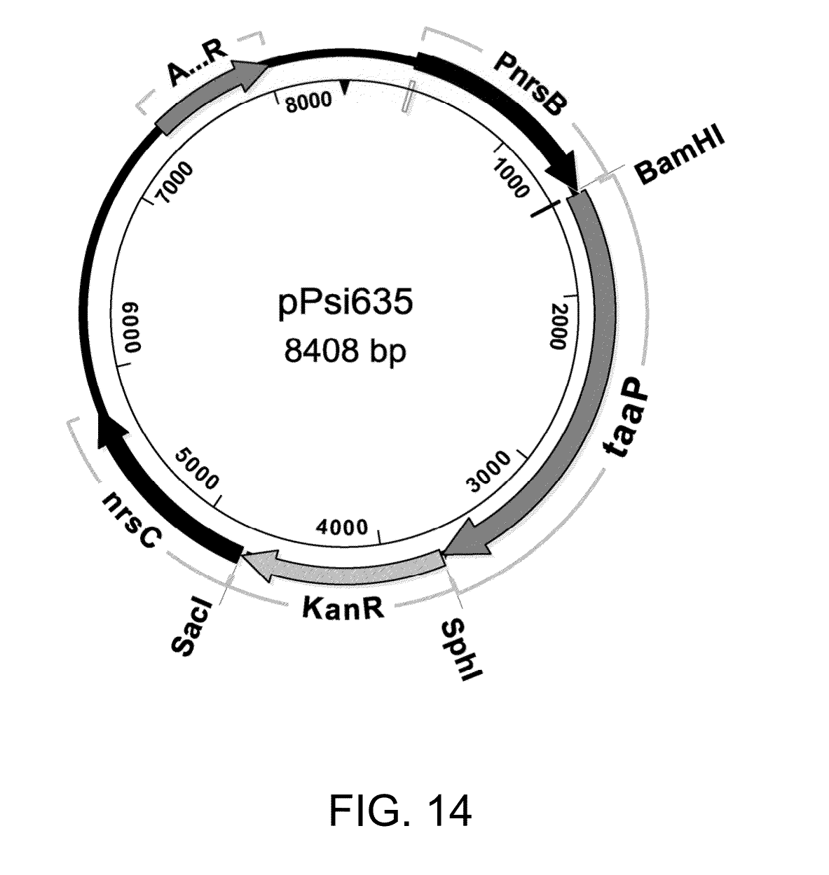

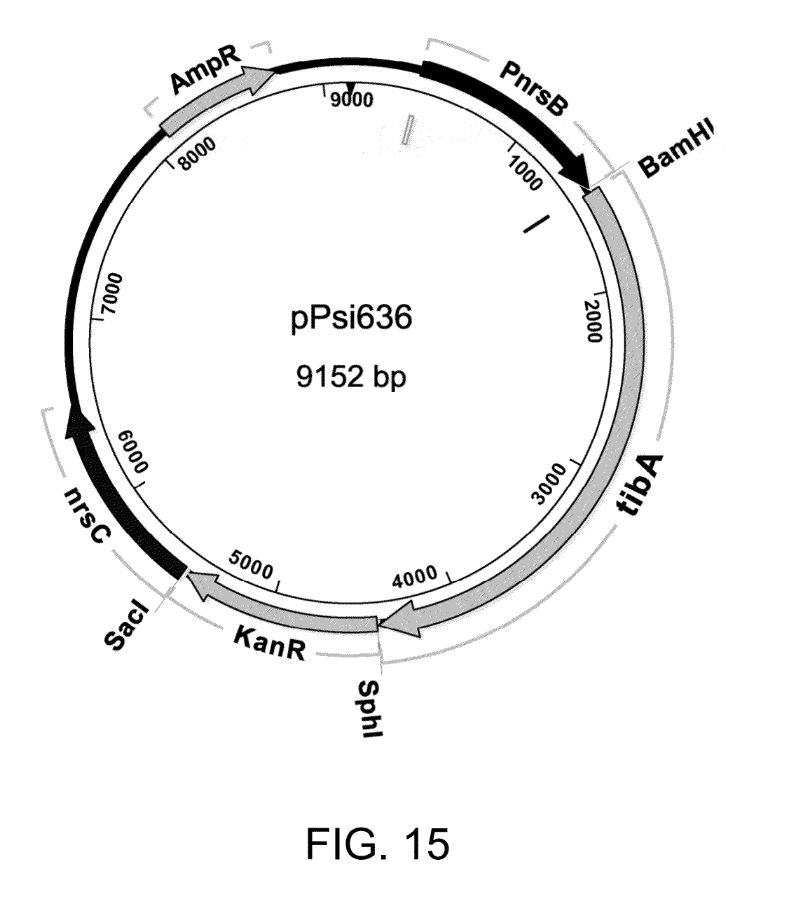

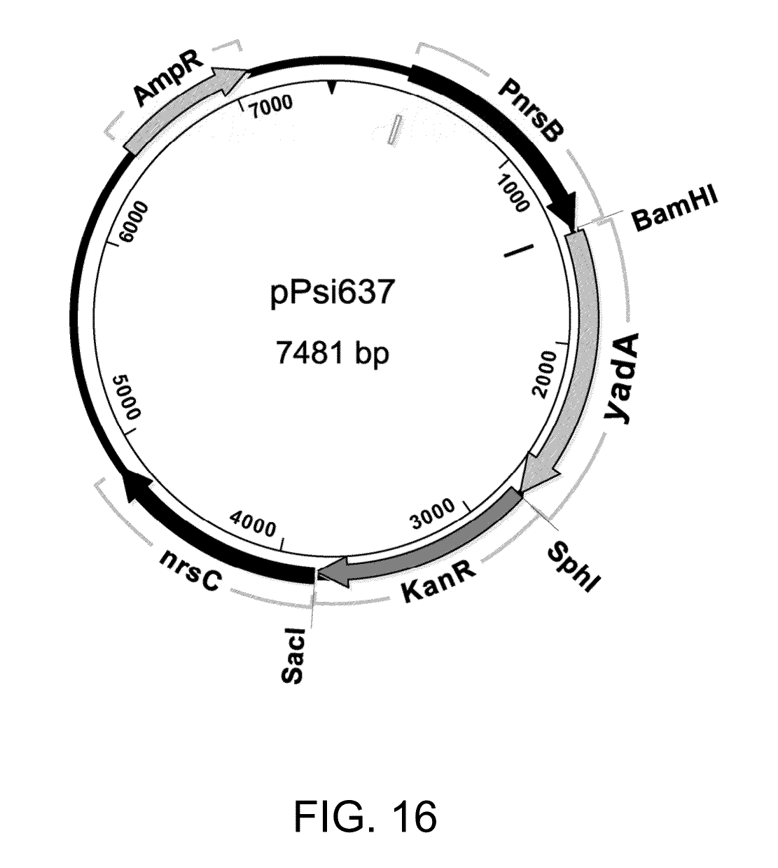

TABLE 1
Strains and Plasmids used in the Invention

| Strain | Genotype |
|---|---|
| SD100 | Synechocystis PCC 6803 |
| SD506 | SD100 Δslr0977::KanR sacB |
| SD536 | SD100 Δslr0977 |
| SD553 | SD100 Δslr0982::KanR sacB |
| SD507 | SD100 Δslr1610::KanR sacB |
| SD565 | SD536 Δsll0574-sll0575::KanR sacB |
| SD562 | SD506::slr0977 |
| SD563 | SD553::slr0982 |
| SD564 | SD507::slr1610 |
| SD342 | SD100 P_{cmpA}::tibA KanR |
| SD500 | Wild type Synechocystis PCC 6803 “Pasteur Collection” |
| SD522 | SD500 Δsll1951::KanR sacB |
| SD1000 | SD500 Δsll1951 |
| SD1014 | SD1000 ΔnrsAC::P_{nrsB} tibA::KanR |
| SD535 | Synechocystis PCC 6803 Jenks variant |
| SD517 | SD535 Δsll1581::KanR sacB |
| SD519 | SD535 Δslr0162-0163::KanR sacB |
| SD523 | SD535 Δsll1951::KanR sacB |
| Plasmids | Description |
| pJet1.2 | general cloning vector- Fermentas #K1231 (pUC19 derivative) |
| pPsbA2ks | Vector source for KanR sacB cassette |
| pSAP2022 | 842-bp coding region of the PMI2122 gene (aipA) cloned |
| | between the I-XhoI sites of pET21A [6] |
| pSAP2025 | 2,225-bp coding region of the PMI2575 gene (taaP) cloned |
| | between the NdeI-XhoI sites of pET21A [6] |
| pψ342 | Suicide vector to insert tibA::Kan^{R} under the control of |
| | PcmpA |
| pψ508 | suicide vector for counterselecting sacB in SD506. (FIG. 5) |
| pψ509 | suicide vector for constructing SD506 (FIG. 6) |
| pψ510 | suicide vector for counterselecting sacB in SD501 |
| pψ511 | suicide vector for constructing SD501 |
| pψ512 | suicide vector for counterselecting sacB in SD507 (FIG. 7) |
| pψ513 | suicide vector for constructing SD507 (FIG. 8) |
| pψ514 | suicide vector for counterselecting sacB in SD565 |
| pψ515 | suicide vector for constructing SD565 |
| pψ541 | suicide vector for constructing SD519 |
| pψ543 | suicide vector for constructing SD523 |
| pψ560 | suicide vector for counterselecting sacB in SD553 (FIG. 9) |
| pψ561 | suicide vector for constructing SD553 (FIG. 10) |
| pψ588 | Suicide vector for the insertion of genes under the control of |
| | PnrsB (FIG. 11) |
| pψ618 | suicide vector for complementing slr0977 mutation in SD506 |
| pψ619 | suicide vector for complementing slr0982 mutation in SD507 |
| pψ620 | suicide vector for complementing slr1610 mutation in SD507 |
| pψ630 | pψ588 + aipA (FIG. 12) |
| pψ634 | Pψ630 + RBS aipA (FIG. 13) |
| pψ635 | pψ630 + RBS tap (FIG. 14) |
| pψ636 | Pψ630 + RBS tibA (FIG. 15) |
| pψ637 | Pψ630 + RBS yadA (FIG. 16) |
| pψ540 | suicide vector for constructing SD517 (FIG. 17) |
| pGEM 3Z | general cloning vector (pUC19 derivative) |

TABLE 2
Primers used in plasmid construction

| | Primer Name and | |
|---|---|---|
| Plasmids | SEQ ID NO | Primer sequence |
| pψ508 & | MLF-1 (SEQ ID NO: | tttatgccactaggttcc |
| pψ618 | 1) | |
| | MLF-2 (SEQ ID NO: | ggatcctttaaaccccatatgcatacttgaggtcaatttttg |
| | 2) | |
| | MLF-3 (SEQ ID NO: | catatggggtttaaaggatcctaaccatggcaacaaac |
| | 3) | |
| | MLF-4 (SEQ ID NO: | ccttcctcaactcttcgttg |
| | 4) | |
| pψ511 & | MLF-9 (SEQ ID NO: | ctactatgggaagatttttg |
| pψ619 | 5) | |
| | MLF-10 (SEQ ID | ggatcctttaaaccccatatgcactcaatccctaggcgag |
| | NO: 6) | |
| | MLF-11 (SEQ ID | catatggggtttaaaggatcctgttagaatgttgagcagg |
| | NO: 7) | |
| | MLF-12 (SEQ ID | tcaagaatttgacccag |
| | NO: 8) | |
| pψ513 & | MLF-17 (SEQ ID | ggtttgaacagaatcaag |
| pψ620 | NO: 9) | |
| | MLF-18 (SEQ ID | ggatcctttaaaccccatatgcggtagcgaaagagccat |
| | NO: 10) | |
| | MLF-19 (SEQ ID | catatggggtttaaaggatccccccaataattctggcaag |
| | NO: 11) | |
| | MLF-20 (SEQ ID | ccaccttagttactccatag |
| | NO: 12) | |
| pψ560 & | MLF-13 (SEQ ID | agtcaactcggaattgt |
| pψ619 | NO: 13) | |
| | MLF-14 (SEQ ID | ggatcctttaaaccccatatgcgaatgactgtatcagacat |
| | NO: 14) | |
| | MLF-15 (SEQ ID | catatggggtttaaaggatccattgcatgaaagctgtaattc |
| | NO: 15) | |
| | MLF-16 (SEQ ID | attagaccgccatcaccg |
| | NO: 16) | |
| | MFL-68 (SEQ ID | caattattttctacacatgtccgatgtaacc |
| | NO: 17) | |
| | MFL-69 (SEQ ID | tgttacatcggacatgtgtagaaaataattg |
| | NO: 18) | |
| pψ342 | TibA-S-S6F1 (SEQ | gccttttttcatagaaaaaatcaaggagaaagttatgaataaggtcta |
| | ID NO: 19) | taacactg |
| | S6F1-A-TibA (SEQ | cagtgttatagaccttattcataactttctccttgattttttctatga |
| | ID NO: 20) | aaaaaggc |
| | TibA-A-KM (SEQ ID | tgcaggtcgactctagctagagttagaagttgattcggaaacc |
| | NO: 21) | |
| | KM-S-TibA (SEQ ID | ggtttccgaatcaacttctaactctagctagagtcgacctgca |
| | NO: 22) | |
| | cmpR-a (SEQ ID | ttctatgaaaaaaggcagacagaaaaattaaataagcgcca |
| | NO: 23) | |
| | cmpA-S (SEQ ID | gaaaaaatcaatcaa agt atgggttcattcaatcgacg |
| | NO: 24) | |
| pψ588 | MLF-117 (SEQ ID | GGCAGGGTTGGTTGACCAAACACAG |
| | NO: 25) | |
| | MLF-112 (SEQ ID | ggccgagctcggccggatccggccACCACCTCAAATTGGGAATTTGTC |
| | NO: 26) | C |
| | MLF-113 (SEQ ID | ggccggatccggccgagctcggccatggagaccacattgctccttttg |
| | NO: 27) | tg |
| | MLF-97 (SEQ ID | TGGCTTGGGCTAGGTATA |
| | NO: 28) | |
| pψ630 | MLF-173 (SEQ ID | aattggatccGTGATTTCATTTATTCTAGATTGTGATGAG |
| | NO: 29) | |
| | MLF-174 (SEQ ID | gagatattatgatattttctgaattgtggcatgcTCACCAGCCATAAG |
| | NO: 30) | CTAATC |
| | MLF-175 (SEQ ID | GATTAGCTTATGGCTGGTGAgcatgccacaattcagaaaatatcataa |
| | NO: 31) | tatctc |
| | MLF-176 (SEQ ID | gagctcttagaaaaactcatcgagcatcaaatgaaac |
| | NO: 32) | |
| pψ634 | MLF-186 (SEQ ID | aattggatccaggagaaagttGTGATTTCATTTATTCTAGATTGTGAT |
| | NO: 33) | GAG |
| | MLF-176 (SEQ ID | gagctcttagaaaaactcatcgagcatcaaatgaaac |
| | NO: 34) | |
| pψ635 | MLF-185 (SEQ ID | ggccGGATCCaggagaaagttATGAAAACGACGGGAGTTAAAG |
| | NO: 35) | |
| | MLF-172 (SEQ ID | ggccgagctcTTACCAGCCCACCGCAAAC |
| | NO: 36) | |
| pψ637 | MLF-181 (SEQ ID | aattggatccaggagaaagttATGACTAAAGATTTTAAGATCAGTGTC |
| | NO: 37) | TCTGCG |
| | MLF-182 (SEQ ID | ggccgcatgcTTACCACTCGATATTAAATGATGCGTTGTACATG |
| | NO: 38) | |
| pψ540 | Up F NheI (Eag) | ggattggctagcattcatagcattcggccgatg |
| | (SEQ ID NO: 39) | |
| | Up R SphI (SEQ ID | Cctggatacgacgcatgccatcatctaag |
| | NO: 40) | |
| | Dn F BamHI (SEQ | GcttgcaGgcataatttttggatccacaagattcc |
| | ID NO: 41) | |
| | Dn R NheI (stop) | Ccaactttaagaaacaatgatgtagtcttagctagcaccagtggttaa |
| | (SEQ ID NO: 42) | act |
| pψ541 | Up PilC Barn F | Ccaatgctctgcggggatccttacgggaagatcc |
| | (SEQ ID NO: 43) | |
| | Up PilC Nhe R | Ggtcagatgattagggggctagcaccgaaaaacttatg |
| | (SEQ ID NO: 44) | |
| | Dn PilC Nhe Eag F | Gccgctagcgttgtgaagagagtacggccgcac |
| | (SEQ ID NO: 45) | |
| | Dn PilC Sph R | gcgGcattcccaagtaaagcatgcgctctttaa |
| | (SEQ ID NO: 46) | |
| pψ543 | UpSLayer | ggGcagtaagcgacgggatccagctcgtttaag |
| | Bam F (SEQ ID NO: | |
| | 47) | |
| | UpSLayer | Ggatttaatctctaaatctgctagctaaagttacgg |
| | NheR (SEQ ID NO: | |
| | 48) | |
| | DnSLaye | Gcacttttcagacacttgctagcggccggggaaaa |
| | NheEagF (SEQ ID | |
| | NO: 49) | |
| | DnSLayer | Ggttggtcttactatagcatgcaggtggtaacgga |
| | SphR (SEQ ID NO: | |
| | 50) | |

TABLE 3
Sequences of exogenous DNA encoding adhesins

| DNA Source, | |
|---|---|
| Accession Number, | |
| and SEQ ID NO | Name and Sequence |
| Y. pseudotuberculosis | yadA: |
| NC 006155.1 | atgactaaagattttaagatcagtgtctctgcggcattaatatctgcgttgttctcatctccatatgc |
| (SEQ ID NO: 51) | atttgccgaggagcccgaggatggcaacgatggtattcctcgtttgtcagcagttcaaataagc |
| | ccaaatgttgatcctaaattgggtgtgggattatatccagcaaaaccaatattacgtcaagaaa |
| | acccaaaattacctccacgaggtccacaaggtccagaaaaaaaaagagctagattagcag |
| | aagcaatacaaccgcaagtactaggcgggctcgatgctcgcgctaagggtatccatagcatt |
| | gcgattggtgctactgctgaagcagcgaaaccagcagcagttgctgtgggcgctggttcaatt |
| | gcaacaggcgttaattctgttgcaattggtcctttaagtaaggcattgggagattcggcagttact |
| | tatggggcaagtagtaccgcccagaaagatggagtagctatcggtgcgagagcatcagcttc |
| | ggatactggtgtcgctgtcggttttaactcgaaagttgatgcacaaaactctgttgccattggaca |
| | ctctagtcacgttgcggcagatcatggttattcaattgcaattggggatctttctaaaactgaccg |
| | agagaatagtgtatccattggtcatgaaagccttaatcgccaattaacacatcttgcggctggc |
| | actaaagacaatgatgcagtgaatgtcgcgcaattaaagaaagaaatggctgaaacattgg |
| | aaaatgcacgtaaagagactttggctcagtctaacgatgttttggatgcggccaaaaaacact |
| | caaatagtgttgccagaacaactttagaaactgctgaagaacatgcaaataaaaaatcagct |
| | gaggcgttagtaagcgctaaagtgtatgcagacagcaattcttctcacacactaaaaactgca |
| | aatagctataccgatgtgactgtaagtagttcgactaagaaagcaatcagtgaatctaatcaat |
| | acacagatcataaattcagtcaacttgacaaccgtttagataaacttgacaaacgagttgaca |
| | aaggtttagccagttcagccgctttaaacagcttgttccagccatatggtgtagggaaagtaaa |
| | ctttactgcaggtgtcgggggatatcgttctagtcaggcattagcaattggttctggctatcgtgta |
| | aatgagagtgtcgcacttaaagccggtgtggcttatgccggttcctcgaatgtcatgtacaacg |
| | catcatttaatatcgagtggtaa |
| E. coli (ATCC 35401) | tibA: |
| NC_017633.1 | atgaataaggtctataacactgtctggaatgaatccacaggaacgtgggtcgtaacttcagaa |
| (SEQ ID NO: 52) | ctgacccgtaaagggggtctacgcccacgacaaatcaaacgtaccgtgctggcagggttga |
| | ttgctggtttgctgatgccgtcgatgcccgctctggcggcagcctatgacaatcagacaattggt |
| | aggggtgaaacaagcaagtccatgcacctgtctgcaggcgacacggcaaaaaacacgac |
| | cattaacagcggtgggaagcagtatgtctccagtggtggcagtgccacgagtaccaccatta |
| | acatcggcggggttcagcatgtttctagcggcggtagtgccactagttccaccatcaacagcg |
| | gagggcaccagcatgtctccagtggtggtagtgccacgaatacaactgttaacaatggtggg |
| | agacagacggtattcagcggcggcagtgccatgggcaccataattaacagcggaggagat |
| | cagtatgtaatcagtggtggcagtgccacaagcgcatctgttaccagcggagcgcgacagttt |
| | gtctccagtggcggcatcgtcaaggcgacttctgttaatagcggtggtcgccagtatgtccgtg |
| | acggcggcagtgccacggacaccgtgcttaacaatactgggcgccagtttgtctccagcggc |
| | ggtagtgccgctaaaactacaattaattccggcggggggatgtatctctacggcggaagtgcc |
| | acgggcacctctatttacaacggcggacgccagtacgtctccagtggtggcagcgccactaa |
| | cacaaccgtttacagcggtggacgtcagcatgtttacatcgatggaaatgtcacagaaacga |
| | ccattacaagcggcggcatgctgcaggtagaggccggtggttcagcctctaaggttatccag |
| | aacagtggcggtgctgtcatcaccaataccagtgcagccgtgagtggtaccaacgataacg |
| | gcagcttcagtatcgctggcggcagtgcagtcaatatgctgctggaaaacggcggatacctg |
| | acggtgtttgacggtcatcaggccagcgatacgatggttggcagtgacggtacgctggatgttc |
| | gcagcggcggtgtgctctacggcaccacaaccctgactgataaaggggcgctcgtcggtga |
| | cgtagtcacaaacgaaggcaacctgtattacctcaacaacagtactgcgactttcaccggtac |
| | cctgacggggaccggtaccctgacacaggaaggcgggaacacccgcttcagcggcctgtt |
| | gtctcaggacggtgggattttccttcagagtggcggggccatgacgatggatgcacttcaggct |
| | aaggctaatgtgacgacgcagtcaggcaccaccctgacgctggataacgggaccatcctga |
| | cggggaacgtggcgggagacagcaccggtgccggtgatatggcggttaaaggtgcctctgt |
| | ctggcatctcgatggtgactccactgtaggcgcattgacgctggataacgggaccgttgatttc |
| | cgtccgtcaacaaccacacgcatgacgccagccttccaggcggtgtcgctggcgctcggga |
| | gcctgtcgggtagcgggacgttccagatgaacacggatatagcatcacataccggagatatg |
| | cttaatgtcgcgggcaatgccagcggtaactttgtgctggatatcaaaaacaccggtctggag |
| | ccagtatctgctggagcaccgcttcaggtggtgcagactggcggcggtgatgccgcgtttacc |
| | ctgaaaggcgggaaagtcgatgccggtacctgggaatacggcctgagcaaggaaaatacc |
| | aactggtatctgaaggctgatactccgcctccagttactccaccgaccaacccggatgcagat |
| | aacccggatgcaggtaacccggatgcaggtaacccggatgcaggtaacccggatgcaggt |
| | aacccggatgctggtaaaccgggaacaggtaagccagatgcaggtacatcgtcgtctccag |
| | tgcgtcgcacaacaaaatcggtcgacgcagtactgggcatggcgacagcaccggcatacgt |
| | cttcaacagcgagctggataatctgcgtttccgtcatggtgatgtgatgcaaaatacccgtgca |
| | ccagggggcgtatggggccgttataccgggtcagacaaccgcattagcggcggggccagc |
| | tcgggctataccctgacccagaacggtttcgaaaccggcgccgatatggtctttgacctgagtg |
| | acagcagtttagcggtaggtactttcttctcttacagcgacaacagcattaaacatgcacgtgg |
| | aggaaagagtaacgttgacagctctggtggtggtctgtacgcgacctggtttgataatgacgg |
| | ctactacgtggacggtgtgctgaaatacaaccgctnaataatgagctgcgtacctggatgagt |
| | gacggcacggcggtgaagggcgattacagccagaacggtttcggcggtagcctcgaagcg |
| | ggcagaaccttcagtctaaatgagaatgcatgggcgcagccatacgtgcggacgactgcatt |
| | tcgggcggataagaaggaaatccgcctgaataacggcatgaaagccagtatcggtgcgac |
| | caaatccctgcaggctgaggctggcctgaagctggggatgaccctggacgttgcaggaaaa |
| | gaggtcaaaccgtacctgtcggcagcggtgagtcatgaatntcggataacaacaaagtccgt |
| | atcaatgacacctatgacttcaggaacgatatctccgggacgaccgggaaatacgggctgg |
| | gtgtcaatgctcaactgaccccaaatgctggtgtctgggctgaagctcgttatgaaaacggtaa |
| | gcagaccgagtcaccaataactggtggcgttggtttccgaatcaacttctga |
| P. mirabilis - | aipA: |
| pSAP2022 [6] | gtgatttcatttattctagattgtgatgagaaaaagaatcatttagcatttattggcaactctgttatc |
| (SEQ ID NO: 53) | cattacaggacgaataacatgctattaaaaaaaatatttttagtgtctttttttattccctacactctctt |
| | acgctaataccttaccaacatcaaatatagataattcactaactcccaaccatgataatcttgcc |
| | cgtttttcactcggtgacaatagtcaagcatccggcagcggtgccactgctataggtatcaata |
| | gccaagccaccaatactcgttcggttgctattggttatgctgcattagccaatgaggaaaatac |
| | ggtttcatttggtaatagtgaagagaaacaaacagcacgactcaccaatgtcagtgaaggta |
| | aaaataatactgatgcagtcaatctggcacaaacaaaagcactacttagtaaaaataaaag |
| | gttaactgatagccaaataaatgtttttagaaatcaaactaataataacttaaacaccatcaaa |
| | aaaaccattcatgagtttgatgattattatcgaagacgacaagcatcaattaccgatgcaattg |
| | aagcacttgataaaaaagttatttcattagaaaaaaaagtctatgcaggtattgcttcctcaattg |
| | ccatgagcaatattccttatcttactcatcatacttttagtggtggcctcggtattagtaattatcgta |
| | ctggcatagcacttgctggaggtatccaatatcagccaaataatgatatagcatttcgatttaact |
| P. mirabilis - | aipA: |
| pSAP2022 [6] | ttaccagcccaccgcaaacccggcgccgatactggtatcgccgccgttattccatgacgcatt |
| (SEQ ID NO: 54) | caggcgcacattggtattggccgtcggcttatactgcaccccggtggctatcgcctgtgcatca |
| | cggtaatgccccatcgccatcccgaacgaaaaacgctcactgttcacatacggaatggaag |
| | atattgcggtcaccccggcaatccccgcattggcccgtttctccacctgactgattttattgtccag |
| | ctgactgaaacgctggtcggtatactgccgggcattgtccagcaccgtgtgggtcacctgccc |
| | gaacgtctcatcggtatagcggtttgcggccgccagggtctgttcctccccctcaatacgggcc |
| | tgctgctcttccgtaatccgcttgtttatcttcgcctcactgtactgaatgctcacatcggtgtagtcc |
| | atggcctgtttcagcgtctgcttatcgccggcaatacgcgcatcccgctcctcatccagttgccct |
| | ttgttgaccgcatccgtttttgcaatgccggccgccacatgcgtcagaaaacgttcctgaccttc |
| | acggccgacggacacgctgttatcccggtcagccaggctgccgtcccccagtgccacactgt |
| | ttttcgccagcgcccgggcatccacacccagtgccacggatttttcgcccagcgccagactctt |
| | atccccgaccgcaacagcctgtttaccctgactcagcgtactctgcgcctcattcagggctttat |
| | ccgccgcctgatattccgcctgtttctgcgccagttcggctttcaccttctccagatcccggatgttt |
| | tccagctcggattgcgtcggacgcaaatcagacaactttgtatccagcgcggagagatcctttt |
| | ccagccgggagagttgcgtttctgcctgcttcagtgcctgatcggtgacggctttttccgccgata |
| | actgctgtttcagctgtgtggcctgtgacgacatggcctgtgtgtgactgagcgcattgtcctgaa |
| | ggtactgatccactttttgtttcagcgcctccgggttggtattcagcccgacggtgttatcccgggg |
| | acggctttccagtaccagcagttgctgtaactctttggcataatccgggctgtccggcgataaac |
| | tgttgaccttatccagctggatcaggtagttcagctgattctcatagcgggtgaaaataccgctg |
| | accatctgattaaaggtgccttcatcgatgttatccagatacagctctgacaggatctgcttcccg |
| | gcctctgtcaccccggtgagcccctgctcaatttgctgccaggcatttttctgataagcctgatac |
| | agattctttttcccgtccgcagaggtgatcagggcgattttggccagctcttcctgataaccggca |
| | acgcccttcagggccgcggtattcaggtgtaaacgggataattgtttcttctgctcgctgtccgtg |
| | accgcatccgaccacagcatcgctttatcactgtggccggcactcagtaccggggacaggg |
| | ccacaccctgatagttggtcgtaatctgagaaccggttttctctttgatggtttcattaaccagagc |
| | acttctggttttcgcttcaatataggcattaacataactgacataaaactgcgtgtcattactgacc |
| | gacgtatcctgctcaacccgttttttcacttcctgagccagacggtcagcctgcccctgctgcca |
| | caacgccacaaaattctcatccacaatctgtgaaaccgcccccagattgcgttcataaatctgt |
| | gccatccggtcagcgacctgctgtgcctgtgtcacctgctgctgttgcgcggtacgctgacccg |
| | ccagggtctcctgttgctgcaacaacggtttcatcgcttcctgacgctccagaatattgctgttcg |
| | ccagcgatttactgtcaaacagggcctgcgtctgcatgtactgctcatgcgcctgcgccctgttc |
| | tgcaacagggcattctgggctttcatcgccgcctgcgcctgttccgccgtcatattgttccccagc |
| | gccatactgccctcaccgacagccgtactgtgctgccccgtggccacggccgtgtcccccagt |
| | gacacgtttttggcctgtaaagcgcaggaataactgacaaccagcgacaaaagcgttacttta |
| | actcccgtcgttttcat |

TABLE 4
Additional SD Strains

| Strain | Genotype | Description |
|---|---|---|
| SD214 | Δaas-23::sacB Km^{R} | Free Fatty Acid (FFA) producing strain. |
| | | Deletion of slr1609 |
| SD216 | Δaas-23::P_{psbA236} tesA136 | FFA producing strain. E. coli ‘tesA gene |
| | | fused with an HA tag is driven by P_{psbA2}, |
| | | with the same orientation of Pₐₐₛ, as the |
| | | deleted aas gene. |
| SD225 | Δaas-23::P_{psbA236} tesA136 | 2nd generation FFA producing strain. |
| | Δ(slr1993-slr1994)-14::P_{cpc39} accB RBS | |
| | accC70 P_{rbc40} accD RBS accA | |
| SD232 | Δaas-23::P_{psbA236} tesA136 | 3rd generation FFA producing strain. |
| | Δ(slr1993-slr1994)-14::P_{cpc39} accB RBS | SD225 + deletion of genes for S-layer + |
| | accC70 P_{rbc40} accD RBS accA | short chain TEs |
| | Δsll1951-15::P_{psbA210} fatB16 (Uc) P_{rbc41} | |
| | fatB262(Ch) | |
| SD243 | Δaas-23::P_{psbA2} tesA136 | 4^{th} generation FFA producing strain. |
| | Δ(slr1993-slr1994)-14::P_{cpc39} accB RBS | Further optimization for C8 C10 shorter |
| | accC70 P_{rbc40} accD RBS accA | chain fatty acid production and deletion of |
| | Δsll1951-15::P_{psbA210} fatB161(Uc) P_{rbc41} | genes for cyanophycin synthesis, on the |
| | fatB262(Ch) | basis of SD232 |
| | Δ(slr2001-slr2002)-17::P_{psbA211} | |
| | fatB262(Ch) | |
| SD249 | Δaas-23::P_{psbA236} tesA136 | 5^{th} generation FFA producing strain. |
| | Δ(slr1993-slr1994)-14::P_{cpc39} accB RBS | PBP2 deletion and Cc FatB1 (C14:0) |
| | accC70 P_{rbc40} accD RBS accA | overproduction |
| | Δsll1951-15::P_{psbA210} fatB161(Uc) P_{rbc41} | |
| | fatB262(Ch) Δ(slr2001-slr2002)- | |
| | 17::P_{psbA211} fatB262(Ch) Δslr1710- | |
| | 19::P_{psbA210} fatB163(Cc) | |
| SD277 | Δaas-23::P_{psbA236} tesA136 | 5^{th} generation FFA producing strain using |
| | Δ(slr1993-slr1994)-14::P_{cpc39} accB RBS | P_{trc} tesA137 |
| | accC70 P_{rbc40} accD RBS accA | |
| | Δsll1951-15::P_{psbA210} fatB161(Uc) P_{rbc41} | |
| | fatB262(Ch) Δ(slr2001-slr2002)- | |
| | 17::P_{psbA211} fatB262(Ch) | |
| | Δslr1710-19::P_{psbA210} fatB163(Cc) | |
| | Δslr2132-22:: P_{trc} tesA137 | |
| SD121 | ΔnrsBAC11::P_{nrsB27} 13 19 15 | Nickel lysis strain. Nickel promoter |
| | | controlling P22 lysis cassette. |
| SD122 | ΔnrsBAC11::P_{nrsB28} S R Rz | Nickel lysis strain. Nickel promoter |
| | | controlling lambda lysis cassette. |
| SD127 | ΔnrsBAC11::P_{nrsB30} 13 S TT | Nickel lysis strain. Nickel promoter |
| | P_{psbA223} 19 15 | controlling holins from P22 and Lambda, |
| | slr1704-50::P_{psbA233} R Rz | and constitutively expressed endolysins |
| | | from P22 and Lambda. |
| SD237 | P_{cmp43}::fol RBS shl RBS | Green Recovery strain using CO₂ limitation |
| | | promoter to control lipolysis genes |
| SD239 | Δaas-23::P_{psbA236} tesA136 | Green Recovery plus FFA secreting strain. |
| | Δ(slr1993-slr1994)-14::P_{cpc39} accB RBS | |
| | accC70 P_{rbc40} accD RBS accA | |
| | Δsll1951-15::P_{psbA210} fatB161(Uc) P_{rbc41} | |
| | fatB262(Ch) | |
| | P_{cmp43}::fol shl RBS | |
| SD254 | Δaas-23::P_{psbA236} tesA136 | Green Recovery plus FFA secreting strain. |
| | Δ(slr1993-slr1994)-14::P_{cpc39} accB | |
| | accC70 P_{rbc40} accD accA Δsll1951 - | |
| | 15::P_{psbA210} fatB161(Uc) P_{rbc41} | |
| | fatB262(Ch) | |
| | P_{cmp43}::fol RBS shl RBS | |
| | P_{sbtA46}::gpl RBS | |
| SD262 | Δaas-23::P_{psbA236} tesA136 | Green Recovery plus FFA secreting strain. |
| | Δ(slr1993-slr1994)-14::P_{cpc39} accB RBS | |
| | accC70 P_{rbc40} accD RBS accA | |
| | Δsll1951-15::P_{psbA210} fatB161(Uc) P_{rbc41} | |
| | fatB262(Ch) P_{cmp43}::fol RBS shl RBS | |
| | P_{sbtA50}::gpl RBS 13 19 15 RBS | |

TABLE 5
Genetically modified strains with altered biofilm formation.

| | | | Reduced |
|---|---|---|---|
| Strain | | Cell Surface | Biofilm |
| Name | Genotype | Phenotype | Formation? |
| SD517 | Δsll1581::Km^{R} sacB | Putative EPS | Yes |
| | | minus | |
| SD519 | ΔpilC::Km^{R} sacB | Apilate | Yes |
| SD523 | Δsll1951::Km^{R} sacB | Lacking | Yes |
| | | S-layer | |
| SD525 | (Intergenic knockout used as a | Wild-type | No |
| | control): intergenic: Km^{R} sacB | | |

### CITED REFERENCES

1. Randall-Hazelbauer L, Schwartz M. Isolation of the Bacteriophage Lambda Receptor from Escherichia coli. Journal of bacteriology 1973 Dec. 1, 1973; 116(3):1436-46.
2. Gehring K, Charbit A, Brissaud E, Hofnung M. Bacteriophage lambda receptor site on the Escherichia coli K-12 LamB protein. Journal of bacteriology 1987 May 1, 1987; 169(5):2103-6.
3. Wang J, Michel V, Hofnung M, Charbit A. Cloning of the J gene of bacteriophage lambda, expression and solubilization of the J protein: first in vitro studies on the interactions between J and LamB, its cell surface receptor. Research in Microbiology 1998; 149(9):611-24.
4. Berkane E, Orlik F, Stegmeier J F, Charbit A, Winterhalter M, Benz R. Interaction of Bacteriophage Lambda with Its Cell Surface Receptor: An in Vitro Study of Binding of the Viral Tail Protein gpJ to LamB (Maltoporin)†. Biochemistry 2006 2006 Feb. 1; 45(8):2708-20.
5. Wang J, Hofnung M, Charbit A. The C-Terminal Portion of the Tail Fiber Protein of Bacteriophage Lambda Is Responsible for Binding to LamB, Its Receptor at the Surface of Escherichia coli K-12. Journal of bacteriology 2000 Jan. 15, 2000; 182(2):508-12.
6. Alamuri P, Löwer M, Hiss J A, Himpsl S D, Schneider G, Mobley H L T. Adhesion, Invasion, and Agglutination Mediated by Two Trimeric Autotransporters in the Human Uropathogen Proteus mirabilis. Infection and Immunity 2010 November 2010; 78(11):4882-94.
7. Liu X, Curtiss R. Nickel-inducible lysis system in Synechocystis sp. PCC 6803. Proceedings of the National Academy of Sciences 2009 Dec. 22, 2009; 106(51):21550-4.
8. Liu X, Fallon S, Sheng J, Curtiss R, 3rd. CO2-limitation-inducible Green Recovery of fatty acids from cyanobacterial biomass. Proc Natl Acad Sci USA 2011 Apr. 26; 108(17):6905-8.
9. Cote J-P, Mourez M. Structure-Function Analysis of the TibA Self-Associating Autotransporter Reveals a Modular Organization. Infection and Immunity 2011 May 1, 2011; 79(5):1826-32.
10. Tahir Y E, Kuusela P, Skurnik M. Functional mapping of the Yersinia enterocolitica adhesin YadA. Identification of eight NSVAIG-S motifs in the amino-terminal half of the protein involved in collagen binding. Molecular microbiology 2000; 37(1):192-206.
11. Roggenkamp A, Neuberger H-R, Flügel A, Schmoll T, Heesemann J. Substitution of two histidine residues in YadA protein of Yersinia enterocolitica abrogates collagen binding, cell adherence and mouse virulence. Molecular microbiology 1995; 16(6):1207-19.
12. Berla B M, Pakrasi H B. Upregulation of Plasmid Genes during Stationary Phase in Synechocystis sp. Strain PCC 6803, a Cyanobacterium. Appl Environ Microbiol 2012 Aug. 1, 2012; 78(15):5448-51.

What is claimed is:

1. A genetically modified phototrophic microorganism capable of controlled adhesion, wherein the microorganism comprises a recombinant nucleic acid construct, wherein the nucleic acid construct comprises an inducible promoter operably-linked to a nucleic acid encoding at least one adhesion protein, wherein the microorganism is capable of regulated expression of the nucleic acid encoding the adhesion protein, and wherein controlled adhesion of the microorganism is selected from the group consisting of:
(a) expressing the nucleic acid encoding the adhesion protein in the microorganism to enhance the adhesion of the microorganism,
(b) reducing the expression of the nucleic acid encoding the adhesion protein to reduce the adhesion of the microorganism, and
(c) reducing the expression of the nucleic acid encoding the adhesion protein to enhance the adhesion of the microorganism.

2. The phototrophic microorganism of claim 1, wherein the microorganism is a Synechocystis PCC sp. 6803 cyanobacterium.

3. The phototrophic microorganism of claim 1, wherein the adhesion protein of (a) is selected from the group consisting of the protein encoded by the tibA nucleic acid sequence of E. coli, the protein encoded by the yadA nucleic acid sequence of Yersinia species, the protein encoded by the aipA nucleic acid sequence of Proteus mirabilis, the protein encoded by the taaP nucleic acid sequence of Proteus mirabilis, the protein Ag43a encoded by the fluA nucleic acid sequence of E. coli.

4. The phototrophic microorganism of claim 1, wherein the adhesion protein of (b) is selected from the group consisting of the protein encoded by the sll1581 nucleic acid sequence of Synechocystis, the protein encoded by the pilC nucleic acid sequence of Synechocystis, and the protein encoded by the sll1951 nucleic acid sequence of Synechocystis.

5. The phototrophic microorganism of claim 1, wherein the adhesion protein of (c) is selected from the group consisting of the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

6. The phototrophic microorganism of claim 1, wherein the nucleic acid comprising an inducible promoter operably-linked to a nucleic acid further comprises a constitutive promoter operably-linked to the nucleic acid.

7. The phototrophic microorganism of claim 1, wherein the inducible promoter is induced by the lack of CO₂ or induced by the addition of Ni.

8. A method of controlling adhesion of a phototrophic microorganism, the method comprising introducing into the microorganism a recombinant nucleic acid construct, wherein the nucleic acid construct comprises an inducible promoter operably-linked to a nucleic acid encoding at least one adhesion protein, wherein the microorganism is capable of regulated expression of the nucleic acid encoding the adhesion protein, and wherein controlling adhesion of the microorganism is selected from the group consisting of:
(a) expressing the nucleic acid encoding the adhesion protein in the microorganism to enhance the adhesion of the microorganism,
(b) reducing the expression of the nucleic acid encoding the adhesion protein to reduce the adhesion of the microorganism, and
(c) reducing the expression of the nucleic acid encoding the adhesion protein to enhance the adhesion of the microorganism.

9. The method of claim 8, wherein the microorganism is a Synechocystis PCC sp. 6803 cyanobacterium.

10. The method of claim 8, wherein the adhesion protein of (a) is selected from the group consisting of the protein encoded by the tibA nucleic acid sequence of E. coli, the protein encoded by the yadA nucleic acid sequence of Yersinia species, the protein encoded by the aipA nucleic acid sequence of Proteus mirabilis, the protein encoded by the taaP nucleic acid sequence of Proteus mirabilis, and the protein Ag43a encoded by the fluA nucleic acid sequence of E. coli.

11. The method of claim 8, wherein the adhesion protein of (b) is selected from the group consisting of the protein encoded by the sll1581 nucleic acid sequence of Synechocystis, the protein encoded by the pilC nucleic acid sequence of Synechocystis, and the protein encoded by the sll1951 nucleic acid sequence of Synechocystis.

12. The method of claim 8, wherein the adhesion protein of (c) is selected from the group consisting of the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis.

13. The method of claim 8, wherein the nucleic acid comprising an inducible promoter operably-linked to a nucleic acid further comprises a constitutive promoter operably-linked to the nucleic acid.

14. The method of claim 8, wherein the inducible promoter is induced by the lack of CO₂ or induced by the addition of Ni.

15. A method of harvesting a phototrophic microorganism, the method comprising:
(a) introducing into the microorganism a nucleic acid comprising an inducible promoter operably-linked to a nucleic acid encoding at least one adhesion protein;
(b) culturing the microorganism;
(c) inducing the promoter to express the nucleic acid encoding the protein;
(d) allowing the microorganisms to autoagglutinate; and
(e) collecting the autoagglutinated biomass.

16. The method of claim 15, wherein the phototrophic microorganism is a Synechocystis PCC sp. 6803 cyanobacterium.

17. The method of claim 15, wherein the adhesion protein is selected from the group consisting of the protein encoded by the tibA nucleic acid sequence of E. coli, the protein encoded by the yadA nucleic acid sequence of Yersinia species, the protein encoded by the aipA nucleic acid sequence of Proteus mirabilis, the protein encoded by the taaP nucleic acid sequence of Proteus mirabilis, the protein Ag43a encoded by the fluA nucleic acid sequence of E. coli, the protein encoded by the slr0977 nucleic acid sequence of Synechocystis, the protein encoded by the slr0982 nucleic acid sequence of Synechocystis, and the protein encoded by the slr1610 nucleic acid sequence of Synechocystis;

18. The method of claim 15, wherein the nucleic acid comprising an inducible promoter operably-linked to a nucleic acid further comprises a constitutive promoter operably-linked to the nucleic acid.

19. The method of claim 15, wherein the inducible promoter is induced by the lack of CO₂ or induced by the addition of Ni.

20. The method of claim 15, wherein the inducible promoter is induced for 2 days.


SEQUENCE LISTING (54 sequences)

SEQ ID NO: 1 - DNA, 18 bp, Artificial Sequence
  Synthesized
tttatgccactaggttcc

SEQ ID NO: 2 - DNA, 42 bp, Artificial Sequence
  Synthesized
ggatcctttaaaccccatatgcatacttgaggtcaatttttg

SEQ ID NO: 3 - DNA, 38 bp, Artificial Sequence
  Synthesized
catatggggtttaaaggatcctaaccatggcaacaaac

SEQ ID NO: 4 - DNA, 20 bp, Artificial Sequence
  Synthetic
ccttcctcaactcttcgttg

SEQ ID NO: 5 - DNA, 20 bp, Artificial Sequence
  Synthetic
ctactatgggaagatttttg

SEQ ID NO: 6 - DNA, 40 bp, Artificial Sequence
  Synthetic
ggatcctttaaaccccatatgcactcaatccctaggcgag

SEQ ID NO: 7 - DNA, 40 bp, Artificial Sequence
  Synthetic
catatggggtttaaaggatcctgttagaatgttgagcagg

SEQ ID NO: 8 - DNA, 17 bp, Artificial Sequence
  Synthetic
tcaagaatttgacccag

SEQ ID NO: 9 - DNA, 18 bp, Artificial Sequence
  Synthetic
ggtttgaacagaatcaag

SEQ ID NO: 10 - DNA, 39 bp, Artificial Sequence
  Synthetic
ggatcctttaaaccccatatgcggtagcgaaagagccat

SEQ ID NO: 11 - DNA, 40 bp, Artificial Sequence
  Synthetic
catatggggtttaaaggatccccccaataattctggcaag

SEQ ID NO: 12 - DNA, 20 bp, Artificial Sequence
  Synthetic
ccaccttagttactccatag

SEQ ID NO: 13 - DNA, 17 bp, Artificial Sequence
  Synthetic
agtcaactcggaattgt

SEQ ID NO: 14 - DNA, 41 bp, Artificial Sequence
  Synthetic
ggatcctttaaaccccatatgcgaatgactgtatcagacat

SEQ ID NO: 15 - DNA, 42 bp, Artificial Sequence
  Synthetic
catatggggtttaaaggatccattgcatgaaagctgtaattc

SEQ ID NO: 16 - DNA, 18 bp, Artificial Sequence
  Synthetic
attagaccgccatcaccg

SEQ ID NO: 17 - DNA, 31 bp, Artificial Sequence
  Synthetic
caattattttctacacatgtccgatgtaacc

SEQ ID NO: 18 - DNA, 31 bp, Artificial Sequence
  Synthetic
tgttacatcggacatgtgtagaaaataattg

SEQ ID NO: 19 - DNA, 56 bp, Artificial Sequence
  Synthetic
gccttttttcatagaaaaaatcaaggagaaagttatgaataaggtctataacactg

SEQ ID NO: 20 - DNA, 56 bp, Artificial Sequence
  Synthetic
cagtgttatagaccttattcataactttctccttgattttttctatgaaaaaaggc

SEQ ID NO: 21 - DNA, 43 bp, Artificial Sequence
  Synthetic
tgcaggtcgactctagctagagttagaagttgattcggaaacc

SEQ ID NO: 22 - DNA, 43 bp, Artificial Sequence
  Synthetic
ggtttccgaatcaacttctaactctagctagagtcgacctgca

SEQ ID NO: 23 - DNA, 41 bp, Artificial Sequence
  Synthetic
ttctatgaaaaaaggcagacagaaaaattaaataagcgcca

SEQ ID NO: 24 - DNA, 38 bp, Artificial Sequence
  Synthetic
gaaaaaatcaatcaaagtatgggttcattcaatcgacg

SEQ ID NO: 25 - DNA, 25 bp, Artificial Sequence
  Synthetic
ggcagggttggttgaccaaacacag

SEQ ID NO: 26 - DNA, 49 bp, Artificial Sequence
  Synthetic
ggccgagctcggccggatccggccaccacctcaaattgggaatttgtcc

SEQ ID NO: 27 - DNA, 50 bp, Artificial Sequence
  Synthetic
ggccggatccggccgagctcggccatggagaccacattgctccttttgtg

SEQ ID NO: 28 - DNA, 18 bp, Artificial Sequence
  Synthetic
tggcttgggctaggtata

SEQ ID NO: 29 - DNA, 40 bp, Artificial Sequence
  Synthetic
aattggatccgtgatttcatttattctagattgtgatgag

SEQ ID NO: 30 - DNA, 54 bp, Artificial Sequence
  Synthetic
gagatattatgatattttctgaattgtggcatgctcaccagccataagctaatc

SEQ ID NO: 31 - DNA, 54 bp, Artificial Sequence
  Synthetic
gattagcttatggctggtgagcatgccacaattcagaaaatatcataatatctc

SEQ ID NO: 32 - DNA, 37 bp, Artificial Sequence
  Synthetic
gagctcttagaaaaactcatcgagcatcaaatgaaac

SEQ ID NO: 33 - DNA, 51 bp, Artificial Sequence
  Synthetic
aattggatccaggagaaagttgtgatttcatttattctagattgtgatgag

SEQ ID NO: 34 - DNA, 37 bp, Artificial Sequence; identical to SEQ ID NO: 32
  Synthetic

SEQ ID NO: 35 - DNA, 43 bp, Artificial Sequence
  Synthetic
ggccggatccaggagaaagttatgaaaacgacgggagttaaag

SEQ ID NO: 36 - DNA, 29 bp, Artificial Sequence
  Synthetic
ggccgagctcttaccagcccaccgcaaac

SEQ ID NO: 37 - DNA, 54 bp, Artificial Sequence
  Synthetic
aattggatccaggagaaagttatgactaaagattttaagatcagtgtctctgcg

SEQ ID NO: 38 - DNA, 44 bp, Artificial Sequence
  Synthetic
ggccgcatgcttaccactcgatattaaatgatgcgttgtacatg

SEQ ID NO: 39 - DNA, 33 bp, Artificial Sequence
  Synthetic
ggattggctagcattcatagcattcggccgatg

SEQ ID NO: 40 - DNA, 29 bp, Artificial Sequence
  Synthetic
cctggatacgacgcatgccatcatctaag

SEQ ID NO: 41 - DNA, 35 bp, Artificial Sequence
  Synthetic
gcttgcaggcataatttttggatccacaagattcc

SEQ ID NO: 42 - DNA, 51 bp, Artificial Sequence
  Synthetic
ccaactttaagaaacaatgatgtagtcttagctagcaccagtggttaaact

SEQ ID NO: 43 - DNA, 34 bp, Artificial Sequence
  Synthetic
ccaatgctctgcggggatccttacgggaagatcc

SEQ ID NO: 44 - DNA, 38 bp, Artificial Sequence
  Synthetic
ggtcagatgattagggggctagcaccgaaaaacttatg

SEQ ID NO: 45 - DNA, 33 bp, Artificial Sequence
  Synthetic
gccgctagcgttgtgaagagagtacggccgcac

SEQ ID NO: 46 - DNA, 33 bp, Artificial Sequence
  Synthetic
gcggcattcccaagtaaagcatgcgctctttaa

SEQ ID NO: 47 - DNA, 33 bp, Artificial Sequence
  Synthetic
gggcagtaagcgacgggatccagctcgtttaag

SEQ ID NO: 48 - DNA, 36 bp, Artificial Sequence
  Synthetic
ggatttaatctctaaatctgctagctaaagttacgg

SEQ ID NO: 49 - DNA, 35 bp, Artificial Sequence
  Synthetic
gcacttttcagacacttgctagcggccggggaaaa

SEQ ID NO: 50 - DNA, 35 bp, Artificial Sequence
  Synthetic
ggttggtcttactatagcatgcaggtggtaacgga

SEQ ID NO: 51 - DNA, 1299 bp, Yersinia pseudotuberculosis
atgactaaagattttaagatcagtgtctctgcggcattaatatctgcgttgttctcatctccatatgcatttgccgaggagcccgaggatggcaacgatggtattcctcgtttgtcagcagttcaaataagcccaaatgttgatcctaaattgggtgtgggattatatccagcaaaaccaatattacgtcaagaaaacccaaaattacctccacgaggtccacaaggtccagaaaaaaaaagagctagattagcagaagcaatacaaccgcaagtactaggcgggctcgatgctcgcgctaagggtatccatagcattgcgattggtgctactgctgaagcagcgaaaccagcagcagttgctgtgggcgctggttcaattgcaacaggcgttaattctgttgcaattggtcctttaagtaaggcattgggagattcggcagttacttatggggcaagtagtaccgcccagaaagatggagtagctatcggtgcgagagcatcagcttcggatactggtgtcgctgtcggttttaactcgaaagttgatgcacaaaactctgttgccattggacactctagtcacgttgcggcagatcatggttattcaattgcaattggggatctttctaaaactgaccgagagaatagtgtatccattggtcatgaaagccttaatcgccaattaacacatcttgcggctggcactaaagacaatgatgcagtgaatgtcgcgcaattaaagaaagaaatggctgaaacattggaaaatgcacgtaaagagactttggctcagtctaacgatgttttggatgcggccaaaaaacactcaaatagtgttgccagaacaactttagaaactgctgaagaacatgcaaataaaaaatcagctgaggcgttagtaagcgctaaagtgtatgcagacagcaattcttctcacacactaaaaactgcaaatagctataccgatgtgactgtaagtagttcgactaagaaagcaatcagtgaatctaatcaatacacagatcataaattcagtcaacttgacaaccgtttagataaacttgacaaacgagttgacaaaggtttagccagttcagccgctttaaacagcttgttccagccatatggtgtagggaaagtaaactttactgcaggtgtcgggggatatcgttctagtcaggcattagcaattggttctggctatcgtgtaaatgagagtgtcgcacttaaagccggtgtggcttatgccggttcctcgaatgtcatgtacaacgcatcatttaatatcgagtggtaa

SEQ ID NO: 52 - DNA, 2970 bp, Escherichia coli
atgaataaggtctataacactgtctggaatgaatccacaggaacgtgggtcgtaacttcagaactgacccgtaaagggggtctacgcccacgacaaatcaaacgtaccgtgctggcagggttgattgctggtttgctgatgccgtcgatgcccgctctggcggcagcctatgacaatcagacaattggtaggggtgaaacaagcaagtccatgcacctgtctgcaggcgacacggcaaaaaacacgaccattaacagcggtgggaagcagtatgtctccagtggtggcagtgccacgagtaccaccattaacatcggcggggttcagcatgtttctagcggcggtagtgccactagttccaccatcaacagcggagggcaccagcatgtctccagtggtggtagtgccacgaatacaactgttaacaatggtgggagacagacggtattcagcggcggcagtgccatgggcaccataattaacagcggaggagatcagtatgtaatcagtggtggcagtgccacaagcgcatctgttaccagcggagcgcgacagtttgtctccagtggcggcatcgtcaaggcgacttctgttaatagcggtggtcgccagtatgtccgtgacggcggcagtgccacggacaccgtgcttaacaatactgggcgccagtttgtctccagcggcggtagtgccgctaaaactacaattaattccggcggggggatgtatctctacggcggaagtgccacgggcacctctatttacaacggcggacgccagtacgtctccagtggtggcagcgccactaacacaaccgtttacagcggtggacgtcagcatgtttacatcgatggaaatgtcacagaaacgaccattacaagcggcggcatgctgcaggtagaggccggtggttcagcctctaaggttatccagaacagtggcggtgctgtcatcaccaataccagtgcagccgtgagtggtaccaacgataacggcagcttcagtatcgctggcggcagtgcagtcaatatgctgctggaaaacggcggatacctgacggtgtttgacggtcatcaggccagcgatacgatggttggcagtgacggtacgctggatgttcgcagcggcggtgtgctctacggcaccacaaccctgactgataaaggggcgctcgtcggtgacgtagtcacaaacgaaggcaacctgtattacctcaacaacagtactgcgactttcaccggtaccctgacggggaccggtaccctgacacaggaaggcgggaacacccgcttcagcggcctgttgtctcaggacggtgggattttccttcagagtggcggggccatgacgatggatgcacttcaggctaaggctaatgtgacgacgcagtcaggcaccaccctgacgctggataacgggaccatcctgacggggaacgtggcgggagacagcaccggtgccggtgatatggcggttaaaggtgcctctgtctggcatctcgatggtgactccactgtaggcgcattgacgctggataacgggaccgttgatttccgtccgtcaacaaccacacgcatgacgccagccttccaggcggtgtcgctggcgctcgggagcctgtcgggtagcgggacgttccagatgaacacggatatagcatcacataccggagatatgcttaatgtcgcgggcaatgccagcggtaactttgtgctggatatcaaaaacaccggtctggagccagtatctgctggagcaccgcttcaggtggtgcagactggcggcggtgatgccgcgtttaccctgaaaggcgggaaagtcgatgccggtacctgggaatacggcctgagcaaggaaaataccaactggtatctgaaggctgatactccgcctccagttactccaccgaccaacccggatgcagataacccggatgcaggtaacccggatgcaggtaacccggatgcaggtaacccggatgcaggtaacccggatgctggtaaaccgggaacaggtaagccagatgcaggtacatcgtcgtctccagtgcgtcgcacaacaaaatcggtcgacgcagtactgggcatggcgacagcaccggcatacgtcttcaacagcgagctggataatctgcgtttccgtcatggtgatgtgatgcaaaatacccgtgcaccagggggcgtatggggccgttataccgggtcagacaaccgcattagcggcggggccagctcgggctataccctgacccagaacggtttcgaaaccggcgccgatatggtctttgacctgagtgacagcagtttagcggtaggtactttcttctcttacagcgacaacagcattaaacatgcacgtggaggaaagagtaacgttgacagctctggtggtggtctgtacgcgacctggtttgataatgacggctactacgtggacggtgtgctgaaatacaaccgctttaataatgagctgcgtacctggatgagtgacggcacggcggtgaagggcgattacagccagaacggtttcggcggtagcctcgaagcgggcagaaccttcagtctaaatgagaatgcatgggcgcagccatacgtgcggacgactgcatttcgggcggataagaaggaaatccgcctgaataacggcatgaaagccagtatcggtgcgaccaaatccctgcaggctgaggctggcctgaagctggggatgaccctggacgttgcaggaaaagaggtcaaaccgtacctgtcggcagcggtgagtcatgaattttcggataacaacaaagtccgtatcaatgacacctatgacttcaggaacgatatctccgggacgaccgggaaatacgggctgggtgtcaatgctcaactgaccccaaatgctggtgtctgggctgaagctcgttatgaaaacggtaagcagaccgagtcaccaataactggtggcgttggtttccgaatcaacttctga

SEQ ID NO: 53 - DNA, 843 bp, Proteus mirabilis
gtgatttcatttattctagattgtgatgagaaaaagaatcatttagcatttattggcaactctgttatccattacaggacgaataacatgctattaaaaaaaatatttttagtgtcttttttattccctacactctcttacgctaataccttaccaacatcaaatatagataattcactaactcccaaccatgataatcttgcccgtttttcactcggtgacaatagtcaagcatccggcagcggtgccactgctataggtatcaatagccaagccaccaatactcgttcggttgctattggttatgctgcattagccaatgaggaaaatacggtttcatttggtaatagtgaagagaaacaaacagcacgactcaccaatgtcagtgaaggtaaaaataatactgatgcagtcaatctggcacaaacaaaagcactacttagtaaaaataaaaggttaactgatagccaaataaatgtttttagaaatcaaactaataataacttaaacaccatcaaaaaaaccattcatgagtttgatgattattatcgaagacgacaagcatcaattaccgatgcaattgaagcacttgataaaaaagttatttcattagaaaaaaaagtctatgcaggtattgcttcctcaattgccatgagcaatattccttatcttactcatcatacttttagtggtggcctcggtattagtaattatcgtactggcatagcacttgctggaggtatccaatatcagccaaataatgatatagcatttcgatttaactcatcaattaatagtgaacaagagcttatttttggcggcggattagcttatggctggtga

SEQ ID NO: 54 - DNA, 2226 bp, Proteus mirabilis
ttaccagcccaccgcaaacccggcgccgatactggtatcgccgccgttattccatgacgcattcaggcgcacattggtattggccgtcggcttatactgcaccccggtggctatcgcctgtgcatcacggtaatgccccatcgccatcccgaacgaaaaacgctcactgttcacatacggaatggaagatattgcggtcaccccggcaatccccgcattggcccgtttctccacctgactgattttattgtccagctgactgaaacgctggtcggtatactgccgggcattgtccagcaccgtgtgggtcacctgcccgaacgtctcatcggtatagcggtttgcggccgccagggtctgttcctccccctcaatacgggcctgctgctcttccgtaatccgcttgtttatcttcgcctcactgtactgaatgctcacatcggtgtagtccatggcctgtttcagcgtctgcttatcgccggcaatacgcgcatcccgctcctcatccagttgccctttgttgaccgcatccgtttttgcaatgccggccgccacatgcgtcagaaaacgttcctgaccttcacggccgacggacacgctgttatcccggtcagccaggctgccgtcccccagtgccacactgtttttcgccagcgcccgggcatccacacccagtgccacggatttttcgcccagcgccagactcttatccccgaccgcaacagcctgtttaccctgactcagcgtactctgcgcctcattcagggctttatccgccgcctgatattccgcctgtttctgcgccagttcggctttcaccttctccagatcccggatgttttccagctcggattgcgtcggacgcaaatcagacaactttgtatccagcgcggagagatccttttccagccgggagagttgcgtttctgcctgcttcagtgcctgatcggtgacggctttttccgccgataactgctgtttcagctgtgtggcctgtgacgacatggcctgtgtgtgactgagcgcattgtcctgaaggtactgatccactttttgtttcagcgcctccgggttggtattcagcccgacggtgttatcccggggacggctttccagtaccagcagttgctgtaactctttggcataatccgggctgtccggcgataaactgttgaccttatccagctggatcaggtagttcagctgattctcatagcgggtgaaaataccgctgaccatctgattaaaggtgccttcatcgatgttatccagatacagctctgacaggatctgcttcccggcctctgtcaccccggtgagcccctgctcaatttgctgccaggcatttttctgataagcctgatacagattctttttcccgtccgcagaggtgatcagggcgattttggccagctcttcctgataaccggcaacgcccttcagggccgcggtattcaggtgtaaacgggataattgtttcttctgctcgctgtccgtgaccgcatccgaccacagcatcgctttatcactgtggccggcactcagtaccggggacagggccacaccctgatagttggtcgtaatctgagaaccggttttctctttgatggtttcattaaccagagcacttctggttttcgcttcaatataggcattaacataactgacataaaactgcgtgtcattactgaccgacgtatcctgctcaacccgttttttcacttcctgagccagacggtcagcctgcccctgctgccacaacgccacaaaattctcatccacaatctgtgaaaccgcccccagattgcgttcataaatctgtgccatccggtcagcgacctgctgtgcctgtgtcacctgctgctgttgcgcggtacgctgacccgccagggtctcctgttgctgcaacaacggtttcatcgcttcctgacgctccagaatattgctgttcgccagcgatttactgtcaaacagggcctgcgtctgcatgtactgctcatgcgcctgcgccctgttctgcaacagggcattctgggctttcatcgccgcctgcgcctgttccgccgtcatattgttccccagcgccatactgccctcaccgacagccgtactgtgctgccccgtggccacggccgtgtcccccagtgacacgtttttggcctgtaaagcgcaggaataactgacaaccagcgacaaaagcgttactttaactcccgtcgttttcat